(19) United States
(12) Patent Application Publication
(10) Pub. No.: US 2013/0022497 A1
(43) Pub. Date: Jan. 24, 2013

(54) CARRIERS FOR PROCESSING POUCHES AND OTHER IRREGULAR CONTAINERS AND OBJECTS

(71) Applicants: Zhijun Weng, Fresno, CA (US); Jeffrey A. Dahl, Fresno, CA (US); Frederick James Wilson, Fresno, CA (US); Fred S. Guerrero, Madera, CA (US); Terry L. Heyliger, Fresno, CA (US); William C. Kreamer, Fresno, CA (US); Kevin Andrew Carlson, Kerman, CA (US)

(72) Inventors: Zhijun Weng, Fresno, CA (US); Jeffrey A. Dahl, Fresno, CA (US); Frederick James Wilson, Fresno, CA (US); Fred S. Guerrero, Madera, CA (US); Terry L. Heyliger, Fresno, CA (US); William C. Kreamer, Fresno, CA (US); Kevin Andrew Carlson, Kerman, CA (US)

(73) Assignee: JOHN BEAN TECHNOLOGIES CORPORATION, Chicago, IL (US)

(21) Appl. No.: 13/554,969

(22) Filed: Jul. 20, 2012

Related U.S. Application Data
(60) Provisional application No. 61/566,524, filed on Dec. 2, 2011.
(60) Provisional application No. 61/510,419, filed on Jul. 21, 2011.

(51) Int. Cl. B25J 11/00; A61L 2/18; B23Q 1/64; B65G 65/00

(52) U.S. Cl. 422/28; 414/800; 414/403; 269/57

(57) ABSTRACT

In accordance with one embodiment of the present disclosure, a carrier for a container for processing the container in a rotary processing system generally includes a carrier body defining at least one compartment for receiving at least one container, wherein the carrier is capable of rolling, and a retention system for maintaining the at least one container in the at least one compartment and restricting movement of the at least one container within the at least one compartment.

### CROSS-REFERENCES TO RELATED APPLICATIONS

This application claims the benefit of U.S. Provisional Application Nos. 61/566,524, filed Dec. 2, 2011, and 61/510,419 filed Jul. 21, 2011, the disclosures of which are hereby expressly incorporated by reference in their entirety herein.

### BACKGROUND

In food processing, various types of continuous sterilization apparatuses have been used for sterilizing cans. While cans are still very popular for containing and processing foods, pouches and other types of containers are also becoming more widely used for food processing. For some food products, pouches are preferred because they are easier to open and easier to dispose of than cans. However, continuous processes, such as continuous rotary sterilization processes are designed for use with cylindrical cans and not for pouches.

Carriers designed for use with continuous sterilization apparatuses that are capable of holding irregularly shaped and/or fragile containers, such as bottles, flat pouches, and other containers have been developed in the past. See, for example, U.S. Pat. Nos. 3,314,560, 4,385,035, and 5,245,916. Although effective, these carriers require assembly of multiple parts, adding complexity to the overall process. Because these carriers have not been optimized for ease of use, there exists a need for optimized carriers for processing irregularly shaped and/or fragile containers, including, but not limited to, pouches, thin-walled cans, bottles, tubs, and other containers.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with one embodiment of the present disclosure, a carrier for a container for processing the container in a rotary processing system is provided. The carrier generally includes a carrier body defining at least one compartment for receiving at least one container, wherein the carrier is capable of rolling, and a retention system for maintaining the at least one container in the at least one compartment and restricting movement of the at least one container within the at least one compartment.

In accordance with another embodiment of the present disclosure, a carrier for a container for processing the container in a rotary processing system is provided. The carrier generally includes a carrier body defining at least one compartment for receiving at least one container, and a removable outer shell for the carrier body.

In accordance with another embodiment of the present disclosure, a method of loading a carrier with a container is provided. The method generally includes obtaining a carrier defining at least one compartment for receiving at least one container and a retention system for maintaining the at least one container in the at least one compartment. The method further includes loading the at least one container in the at least one compartment, and activating the retention system to restrict the movement of the at least one container within the at least one compartment.

In accordance with another embodiment of the present disclosure, a method of loading a carrier with a container is provided. The method generally includes obtaining a carrier defining first and second compartments hingedly coupled to one another between open and closed positions and defining a cylinder when in the closed position. The method further includes placing the first and second compartments in the open positions and receiving at least one container in either of the first and second compartments, restricting the movement of the at least one container within either of the first and second compartments, and hingedly coupling the first and second compartments in the closed position to define a carrier.

In accordance with another embodiment of the present disclosure, a method of processing a container in a rotary processing system is provided. The method generally includes placing at least one container in a carrier, and receiving the at least one carrier in the rotary system.

In accordance with another embodiment of the present disclosure, a system for unloading a container from a carrier having at least one compartment for receiving at least one container and a retention system for maintaining the at least one container in the at least one compartment is provided. The system generally includes a conveyor assembly for conveying the carrier in a controlled path, and an unloading assembly for opening the at least one compartment.

In accordance with another embodiment of the present disclosure, a method for unloading a container from a carrier is provided. The method generally includes receiving on a conveyor assembly, a carrier having at least one compartment for receiving at least one container and a retention system for maintaining the at least one container in the at least one compartment, wherein the conveyor assembly is configured for conveying the carrier in a controlled path, and opening the at least one compartment.

### DESCRIPTION OF THE DRAWINGS

The foregoing aspects and many of the attendant advantages of this disclosure will become more readily appreciated by reference to the following detailed description, when taken in conjunction with the accompanying drawings, wherein:

FIG. 1 is a front view of a rotary sterilizer, which is in the process of rotating two pouch carriers that have been designed in accordance with one embodiment of the present disclosure;

FIG. 2 is an isometric view of the pouch carrier shown in FIG. 1, showing pouches being inserted into the pouch carrier;

FIG. 3 is an isometric view of a pouch carrier in accordance with another embodiment of the present disclosure;

FIGS. 4 and 5 are side cross-sectional views of the pouch carrier of FIG. 3, showing pouches in the pouch sleeves;

FIGS. 6A-15B are various views of various examples of sleeve retention mechanisms for pouch carriers in accordance with embodiments of the present disclosure;

FIGS. 16A-16C are top views of various examples of sleeve shapes and configurations for pouch carriers in accordance with embodiments of the present disclosure;

FIG. 17 is a top view of trapezoidal sleeve shape shown in FIG. 16A, combined with a sleeve retention mechanism, similar to that shown in FIGS. 14A and 14B, in accordance with one embodiment of the present disclosure;

FIGS. 18-20 are directed to various designs for rotary sterilizers in accordance with embodiments of the present disclosure;

FIGS. 21-23 are various views of systems for processing pouch carriers in accordance with embodiments of the present disclosure;

FIG. 24 is an isometric view of a bottle carrier in accordance with another embodiment of the present disclosure;

FIG. 25 is an isometric view of a tub carrier in accordance with another embodiment of the present disclosure;

FIG. 26 is an isometric view of a laminate container carrier in accordance with another embodiment of the present disclosure;

FIG. 27 is an exploded view of the carriers shown in FIGS. 24 and 25, showing interchangeability between carrier sleeve portions and outer shell portions;

FIGS. 28-29B are isometric view of carriers in accordance with other embodiments of the present disclosure; and

FIGS. 30-38 are views of various embodiments of carriers designed for holding fragile containers, such as thin-walled cans and plastic bottles.

### DETAILED DESCRIPTION

The detailed description set forth below in connection with the appended drawings, where like numerals reference like elements, is intended as a description of various embodiments of the disclosed subject matter and is not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result.

In the following description, numerous specific details are set forth in order to provide a thorough understanding of exemplary embodiments of the present disclosure. It will be apparent to one skilled in the art, however, that many embodiments of the present disclosure may be practiced without some or all of the specific details. In some instances, well-known process steps have not been described in detail in order not to unnecessarily obscure various aspects of the present disclosure. Further, it will be appreciated that embodiments of the present disclosure may employ any combination of features described herein.

Embodiments of the present disclosure are directed to carriers for irregularly shaped and/or fragile containers or other objects, such as pouches, bottles, thin-walled cans, tubs, fiberboard containers, fiberboard and plastic laminate containers (such as TETRA RECART® containers), and other objects. The various carriers and methods described herein are particularly useful in cooking and sterilization processes using rotary systems, such as rotary sterilizers. However, it should be appreciated that the carriers described herein may also be useful in other non-food-related rotary processes.

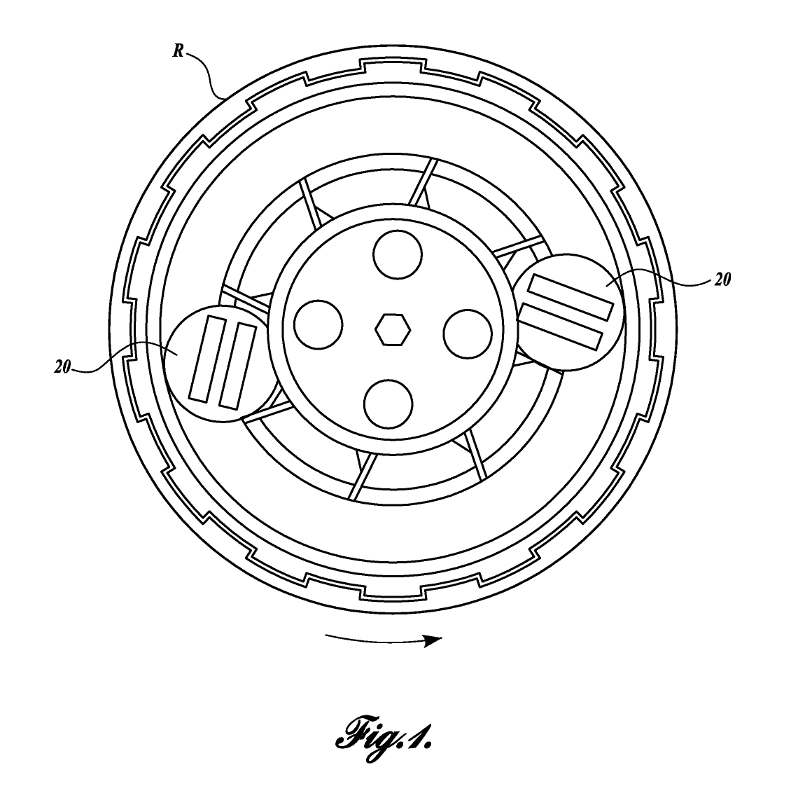

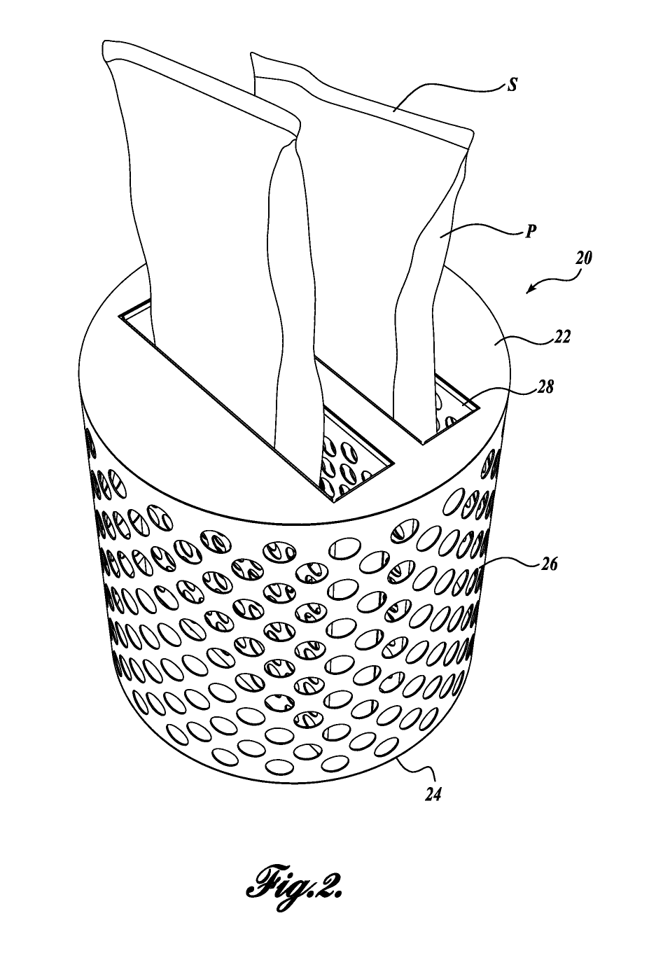

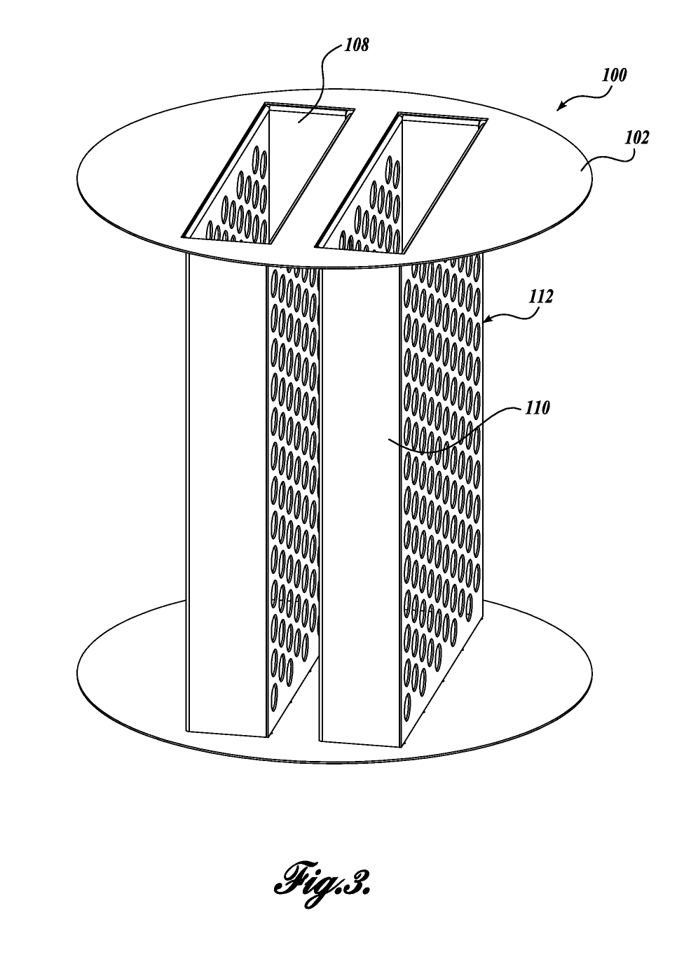

Referring to FIGS. 1 and 2, an exemplary pouch carrier 20 in accordance with embodiments of the present disclosure is shown. In the illustrated embodiment of FIG. 1, the pouch carrier 20 is designed to behave like a cylindrical can when used in a rotary sterilizer R (see also FIGS. 18-20 for other exemplary configurations and designs for rotary systems, such as rotary sterilizers). As can be seen in FIG. 2, the pouch carrier 20 has first and second circular ends 22 and 24 and a cylindrical outer shell portion 26. The first end 22 includes a plurality of sleeve openings 28 for receiving pouches P (see FIG. 3).

The pouch carrier 20 is preferably a one-piece assembly for easily receiving a pouch P without requiring assembly or disassembly. For example, the pouch carrier 20 does not have a clam shell structure, a tray assembly, or a capsule assembly, for enclosing a container on all sides after the container has been received by the carrier, as described in respective U.S. Pat. Nos. 5,245,916, 4,385,035, and 3,314,560 for previously developed technologies, the disclosures of which are hereby expressly incorporated by reference herein.

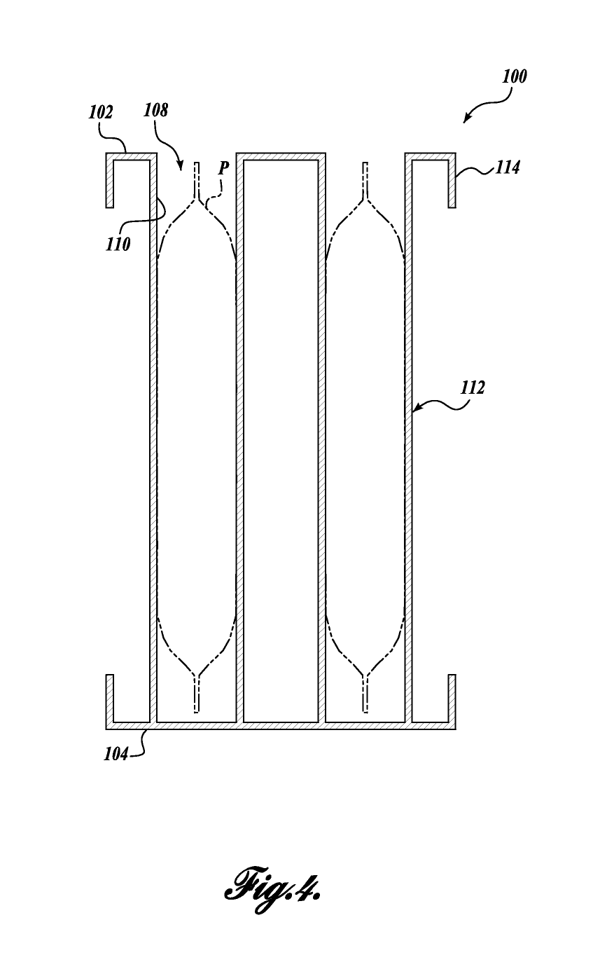

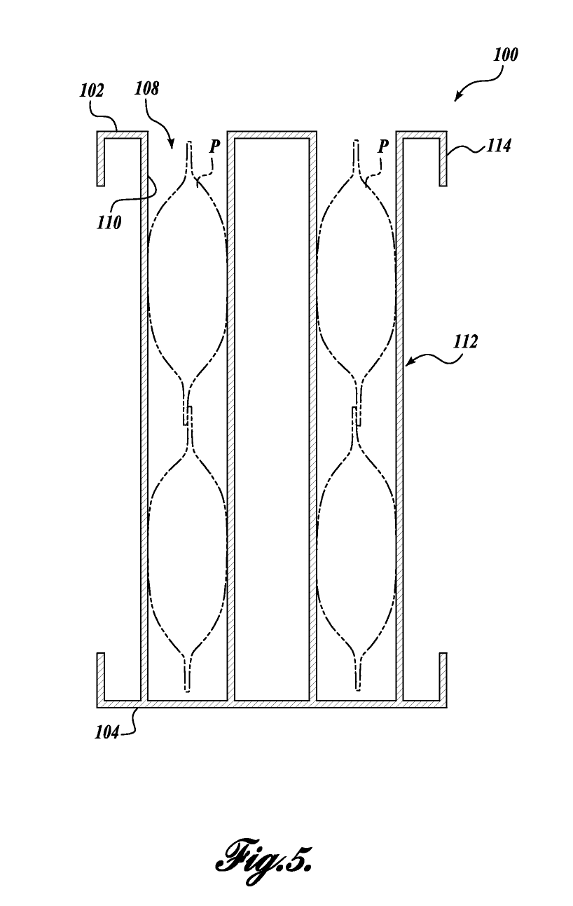

Referring now to FIGS. 3-5, an alternate embodiment of a pouch carrier 100 is shown. This embodiment is substantially similar to the pouch carrier 20 shown in FIGS. 2 and 3, except for differences regarding the outer shell. In that regard, in the illustrated embodiment of FIGS. 3-5, the carrier 100 has no outer shell, exposing the body portion 112 of the carrier, which defines the structure of the pouch sleeves 110. Like the pouch carrier 20 shown in FIG. 2, the pouch carrier 100 shown in FIGS. 3-5 is also suitably configured as a one-piece assembly. Although the pouch carrier 100 of FIGS. 3-5 does not include an outer shell, rolling action of the carrier 100 is enabled by the first and second circular ends 102 and 104 (which may include an optional outer lip 114 extending from the outer perimeter of the first and second circular ends 102 and 104, see FIGS. 4 and 5).

As seen in the illustrated embodiments, the carriers may include perforated portions. In that regard, the walls of the outer shell portion 26 in FIG. 2 and body portion 112 in FIG. 3 are shown as being perforated. Such perforations allow for efficient and consistent heat transfer to or from the pouch P during processing, for example, steam may travel through the walls of the carrier 100 to reach the pouch P. In accordance with embodiments of the present disclosure, suitable perforations may be about 0.20 inches in diameter. However, other sizes are also within the scope of the present disclosure. It should further be appreciated that, in accordance with embodiments of the present disclosure, the body portion 112 need not be made from a perforated material.

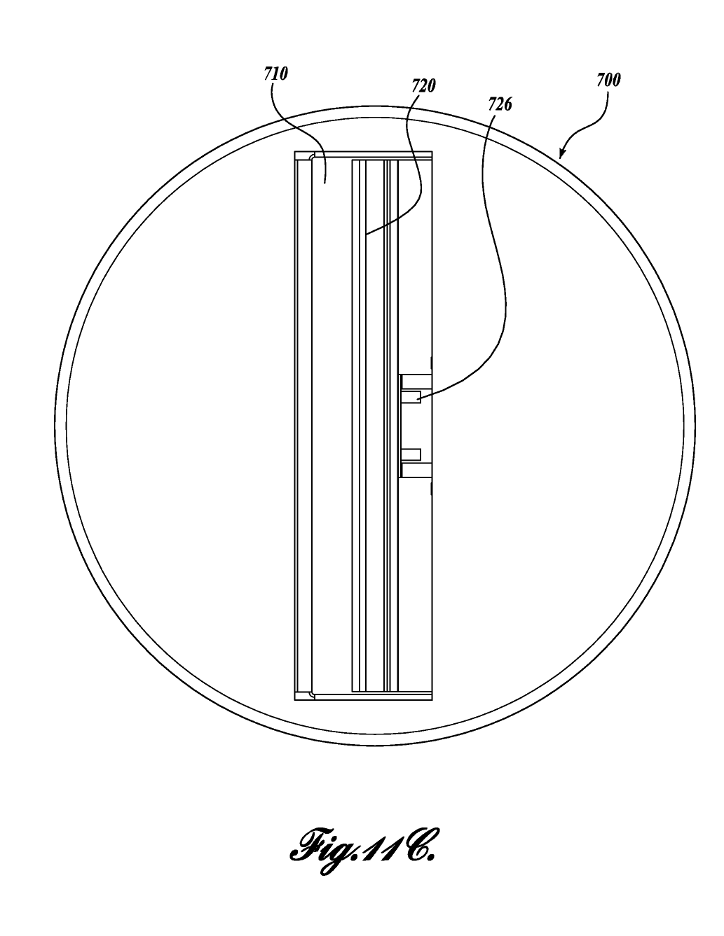

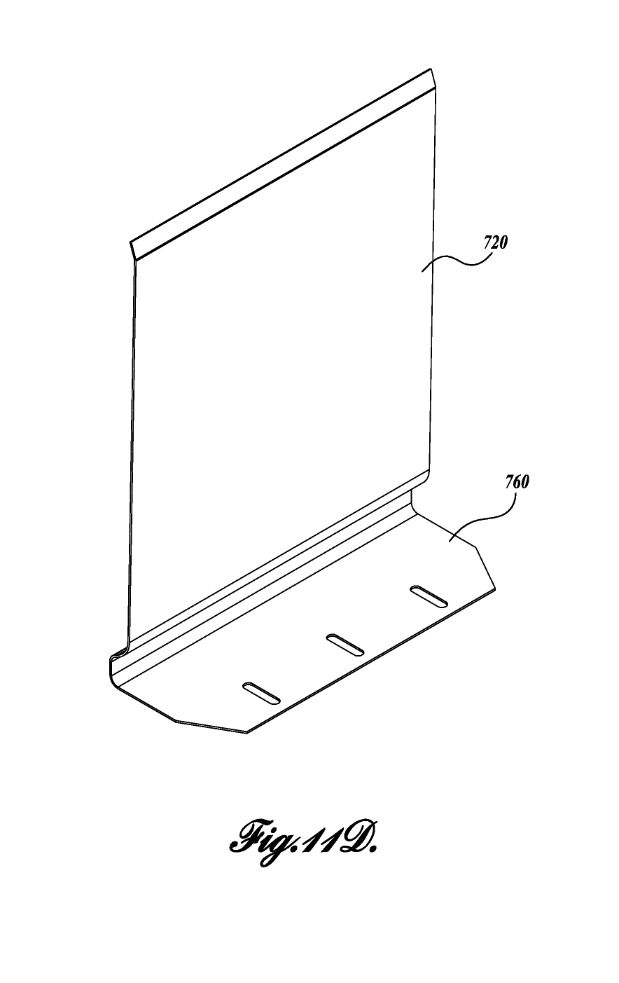

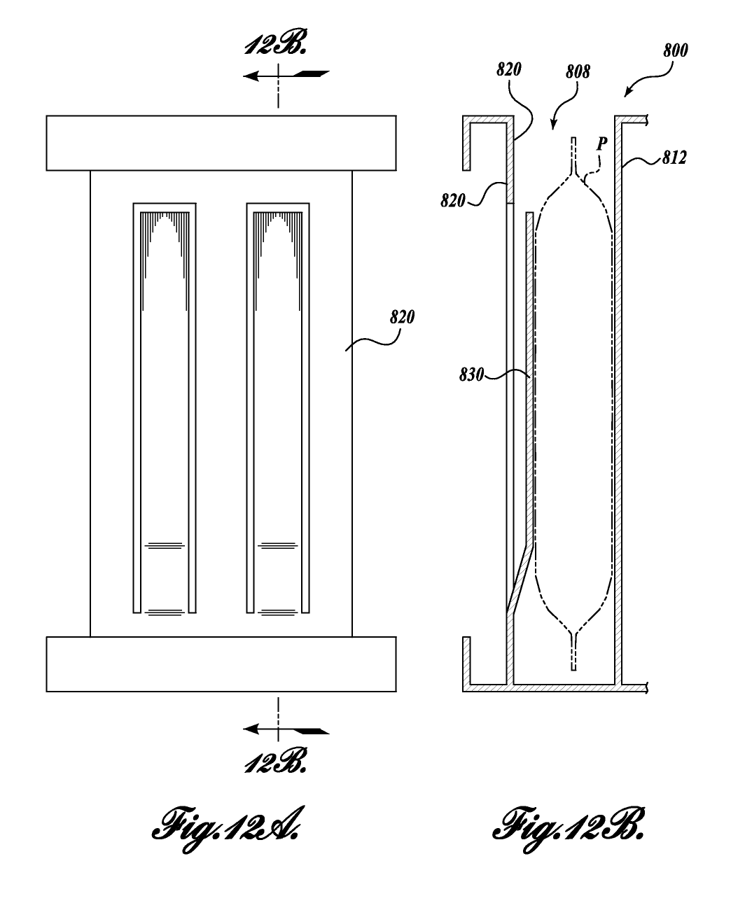

As shown in the illustrated embodiments of FIGS. 1-5, the pouch carriers 20 and 100 include two pouch sleeves. However, it should be appreciated that the pouch carriers 20 and 100 may include any number of pouch sleeves to accommodate various shapes and sizes of pouches. Further, although shown in the illustrated embodiments as having rectangular shaped cross-sections, the pouch sleeves may have other suitable cross-sectional shapes. For example, suitable cross-sectional shapes may include, but are not limited to, trapezoidal, having tapered ends, and curved, as described in greater detail below with reference to FIGS. 16A-16C.

Returning to FIG. 2, the pouches P to be carried by the pouch carriers are generally made of film laminated from layers of plastic and/or foil. The pouches P may be designed for containing various food products, including liquid products or more solid products, such as soups or stews. The pouches P generally each have a thickness when filled. Seams S around the outer peripheral edge of the pouch P contain the products. As seen in the illustrated embodiment, the pouches may be rectangular shaped or another suitable shape as deemed to be desirable for specific food products, processing parameters, or handling requirements.

Referring to the cross-sectional view of the body portion 112 in FIGS. 4 and 5, the sleeve openings 108 and the sleeves 110 are sized to allow for gentle and quick loading of the pouches P without damage to the pouches P. In that regard, the sleeve openings 108 and the sleeves 110 themselves are suitably sized and shaped to receive pouches P. The sleeves 110 may be designed to adjust the contents of the pouches P to provide for substantially even pouch thickness along the length of the pouch. In that regard, the dimensions of the sleeves 110 may provide enough resistance and gentle “squeeze” on the pouch contents to effectively retain the pouches P within the sleeves 110, even during movement of the pouch container 100 (see, e.g., movement in the rotary sterilizer shown in FIG. 1). Referring to FIG. 4, the pouch carrier 100 is shown holding two larger sized pouches P. Referring to FIG. 5, the pouch carrier 100 is shown holding four smaller sized pouches P in a stack of two in each sleeve 110.

After pouches P have been received in the sleeves 110, the sleeves 110 may include a retention system to further retain the pouches P within the sleeves 110 and/or to more evenly distribute the pouch contents within the pouch P for more even pouch thickness distribution within the sleeve. Suitable retention systems are used to minimize pouch movement when the pouch carrier 100 moves to minimize scuffing or wrinkling of the pouches P. Minimizing pouch scuffing and wrinkling not only preserves pouch aesthetics, but also pouch integrity. Moreover, retention systems that provide more even pouch thickness distribution within the sleeve 110 will provide for more consistent heat transfer to the contents in the pouches P. As a one-piece assembly, suitable retention systems are designed so that they do not separate from the body portion 112 of the carrier 100.

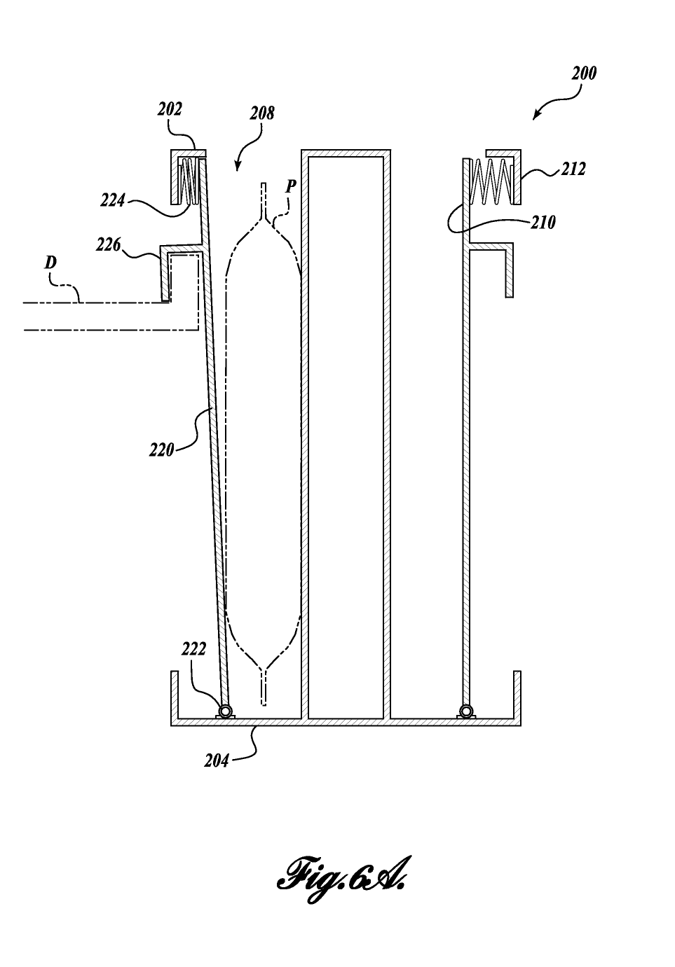

With reference to FIGS. 6A-17, various embodiments of pouch loading and retention devices are shown. For example, referring to FIG. 6A, the outer sleeve wall 220 may be movable from its normally biased closed position (see right side sleeve for normal biased position) to open the sleeve 210 beyond its normal size to allow a user to easily slide a pouch P into the sleeve 210 (see left side sleeve for opened position). When the outer sleeve wall 220 is released, it biases back toward its normal position to gently “squeeze” the pouch P.

In the illustrated embodiment of FIG. 6A, the outer pouch wall 220 is coupled by a hinge 222 to the second end 204 of the body portion 212. A biasing member 224 (such as a compression coil spring or other type of biasing member) adjacent the first end 202 of the body portion 212 is normally biased in the closed position (such as the parallel wall position shown in FIG. 6A). When loading and unloading, a pulling device D, such as a stationary cam or a pull actuator, can be used to engage with a handle 226 to pull the outer wall 220 to the opened position. After the pouch P has been loaded, the bulge of the pouch P may resist the outer wall 220 as it extends to the closed position, thereby holding the pouch in a gently “squeezed” position.

Of note, the opening 208 in the first end 202 of the body portion 212 is larger than the normally biased closed position to allow for loading of the pouch when the outer pouch wall 220 is in the opened position. Referring to FIG. 6B, the opening 208 in the first end 202 of the body portion 212 may include an optional lip 214 extending inwardly from the outer wall 220 to further assist with retention of the pouch P in the sleeve 210.

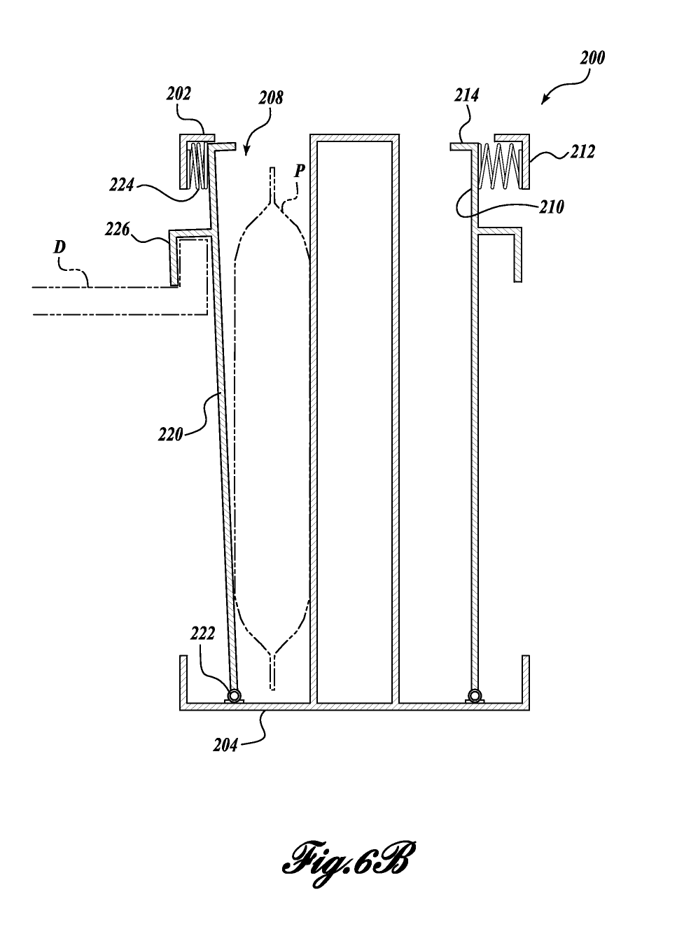

Referring to FIGS. 7A and 7B, respective side isometric and top views of an exemplary hinged retention carrier 300 (similar to the embodiments in FIGS. 6A and 6B) are shown. The sleeves 310 (see FIG. 7B) includes first and second upright sleeve walls 330 and 332. The first sleeve wall 330 is rotatable relative to the second end 304 of the carrier 300 about a hinge (not shown, but positioned along the bottom of wall 330). Biasing members 324 (shown as coil springs) are attached to stationary brackets 334. The biasing members 324 bias the first sleeve wall 330 to a closed position (see FIG. 7B). Handle 336 (see FIG. 7A) can be grasped or otherwise used to pull the rotatable first sleeve wall 330 to an open position to insert or release the pouch P (shown in the left side FIG. 7B to be contained within the sleeve 310). In the illustrated embodiment, handle 336 is pulled through a hole in a stationary wall 340.

The carrier 300 may further include an adjustable stop (not shown) to limit the travel distance of the first sleeve wall 330 to the closed position. As a non-limiting example, the adjustable stop may be a bolt or threaded stud attached to the first sleeve wall 330 and passing through a hole 338 in the stationary wall 340. The adjustable stop may, for example, includes adjustable nuts which rest against the stationary wall 340 or at least one of the stationary brackets 334 to limit the travel distance of the second sleeve relative to the stationary wall 340 or at least one of the stationary brackets 334.

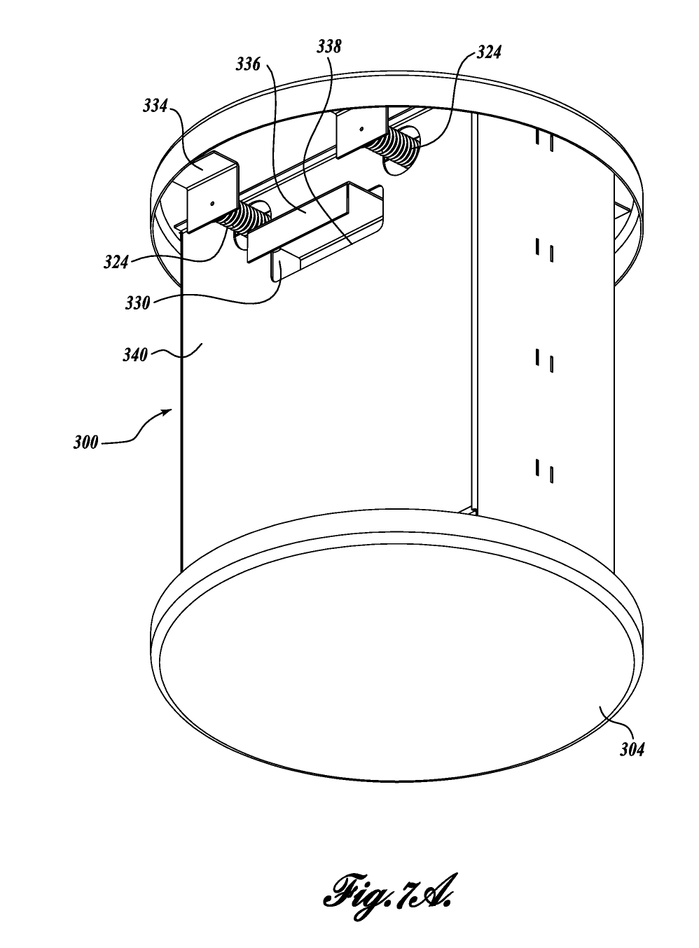

Referring now to FIG. 8, another embodiment of a hinged retention carrier 400 is shown (similar to the embodiments of FIGS. 6A-7B). The carrier 400 includes a position limiter assembly 440 to limit the travel distance of the second sleeve wall 420 coupled to hinge 422. The position limiter assembly 440 includes a position limiting device 442 that extends outwardly from the outer sleeve wall 420, and a stationary surface 412 to which the position limiting device 442 may releasably couple.

In the illustrated embodiment of FIG. 8, the position limiting device 442 includes a first contact surface 446 and a second contact surface 448, which are capable of coupling with the stationary surface in two different sleeve positions, for example, a processing position and a loading/unloading position (e.g., compare positioning of right side sleeve 410 with left side sleeve 410 in FIG. 8). In the illustrated embodiment, the stationary surface 412 includes a substantially vertical wall 450, for example, projecting downward from the first end 402 of the carrier 400. The stationary surface 412 further includes a slot or hole 452 through which the position limiting device 442 is received.

It should be appreciated that the position limiting device 442 may be made from a flat spring material, such that it is capable of bending or flexing when in either of the first and second sleeve positions (e.g., compare positioning of right side sleeve 410 with left side sleeve 410 in FIG. 8). In that regard, looking at the right side sleeve 410, the first contact surface 446 of the position limiting device 442 passes through slot or hole 452 and engages with an upper outer surface of the substantially vertical wall 450 of the stationary surface 412. This engagement maintains the sleeve 410 in a position for pouch retention to a width Wt and prevents the outer sleeve wall 420 from collapsing inwardly.

Now looking to the left side sleeve 410, the second contact surface 448 of the position limiting device 442 engages with a lower outer surface of the substantially vertical wall 450 of the stationary surface 412. The transition from the first position (see right side sleeve 410) to the second position (see left side sleeve 410) is achieved by pulling the outer sleeve wall 420 outwardly (for example, using pulling device D to hook downwardly extending flange 426) from the first position to the second position. As the position limiting device 442 moves outwardly through hole 452, the second contact surface 448 engages with the lower outer surface of the substantially vertical wall 450 of the stationary surface 412. In the second position, the sleeve 410 will remain open for loading or unloading.

The second contact surface 448 in the illustrated embodiment, is shown as a catch or tab with a cam ramp surface that is configured to engage with a lower edge of slot 452. The cam ramp surface allows for the transition from the first position to the second position while minimizing the risk of the position limiting device 442 catching en route. To transition from the second position back to the first position, the position limiting device 442 is lifted upwards to disengage the second contact surface 448 from the lower outer surface of the substantially vertical wall 450 of the stationary surface 412.

In the illustrated embodiment, the second sleeve wall 420 is normally biased in the closed position, with the wall being biased to a tapered position, having a smaller nominal sleeve width Wt at the top of the sleeve than at the bottom of the sleeve Wb. The inventors have found that a sleeve having a tapering width (as shown narrower at the top Wt and wider at the bottom Wb) helps to maintain the pouch (not shown) in the sleeve. It should further be appreciated that the sleeve closed position can be designed and dimensioned (depending on the size and shape of the specific pouch to be received) to substantially evenly distribute the contents in the pouch by bearing against a substantial surface area of the pouch. Such substantially even distribution can provide for more even heat transfer to the contents in the pouch for predictable and repeatable thermal processing.

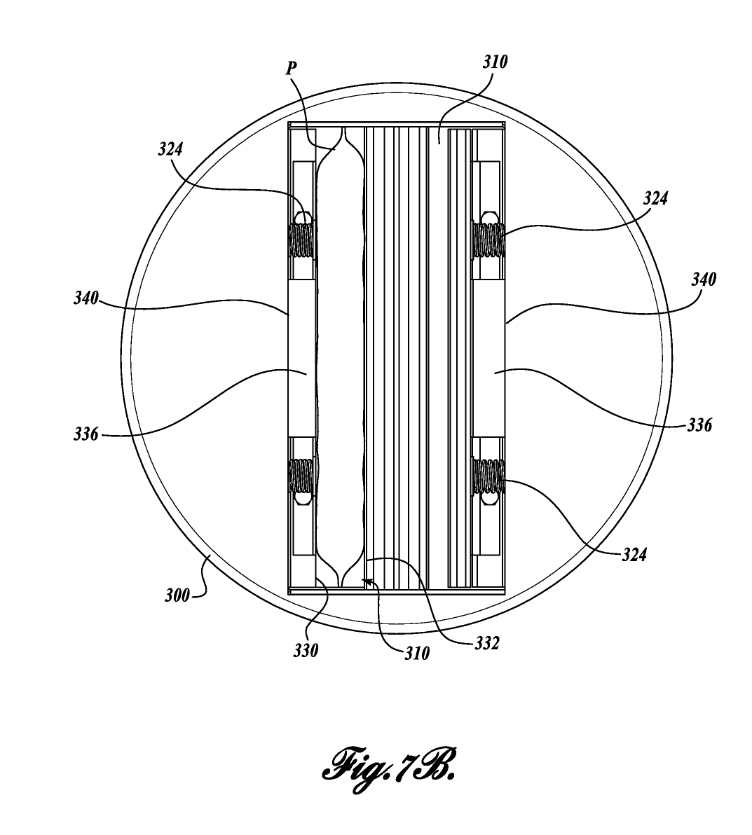

Referring now to FIG. 9, another embodiment of a pouch retention device is shown. Like the embodiments shown in FIGS. 6A-8, the outer sleeve wall 520 in the illustrated embodiment of FIG. 9 may also be movable from its normally biased closed position (see right side sleeve for normally biased closed position) to open the sleeve 510 beyond its normal size to allow a user to easily slide a pouch P into the sleeve 510 (see left side sleeve for opened position). When the outer sleeve wall 520 is released, it biases back toward its normal position to gently “squeeze” the pouch P. However, the outer wall 520 in the illustrated embodiment of FIG. 9 is not hinged like the outer walls 220, 320, and 420 in the illustrated embodiments of FIG. 6A-8. In that regard, outer wall 520 in FIG. 9 acts like a leaf spring and is bendable from its normal closed position to its opened position.

Now referring to FIG. 10A, a position limiter assembly 640 for a leaf spring-type carrier 600 is shown. In the illustrated embodiment, a carrier 600 is shown having two sleeves 610, one on the left side in a normal open loading/unloading position, and one on the right side in a closed processing position. Referring first to the open sleeve 610 on the left side of the drawing, the second sleeve wall 620 is biased in the open position and the position limiter assembly 640 extends unengaged through a slot or hole 652 in the substantially vertical wall 650 of the stationary surface 612. In the embodiments of FIG. 8, the substantially vertical wall 450 of the stationary surface 412 extends for only a portion of the distance between the top and bottom ends of the carrier 400. However, in the illustrated embodiment of FIG. 10A, the substantially vertical wall 650 of the stationary surface 612 extends for the entire distance between the top and bottom ends of the carrier 600.

Referring to FIGS. 10A and 10B, a stopping device 648 (shown as a finger) may extend from the bottom surface of the position limiter assembly 640 to prevent inadvertent opening and control the closing width of the sleeve 610.

To close the open sleeve 610 on the left side of the drawing, the position limiter assembly 640 is pushed to the right until the contact surface 648 (shown as a pierced tab) disengages the lower inner surface of the substantially vertical wall 650 of the stationary surface 612 (for example, similar to the position shown on the rights side of the drawing, but in minor image). Referring now to the closed sleeve 610 on the right side of the drawing, the contact surface 648 engages the lower inner surface of the substantially vertical wall 650 of the stationary surface 612, and the sleeve 610 is maintained in a closed position. To open the closed sleeve 610 on the right side of the drawing, the position limiter assembly 640 is pushed up to release the engagement of the contact surface 648 and the lower inner surface of the substantially vertical wall 650 of the stationary surface 612. The second sleeve wall 620 then moves outwardly toward the substantially vertical wall 650 to rest in an open position (for example, similar to the position shown on the left side of the drawing, but in mirror image).

It should be appreciated that the carriers described herein are designed with robust position limiting devices, such that the carriers will remain in either of their designated open or closed positions as they travel through handling and processing shocks. Moreover, it should be appreciated that the carriers may be designed to have one or more position limiting assemblies that are capable of stopping in more than two (e.g., open and closed) positions. For example, the carriers may have different open positions designed for different sized pouches. Although the embodiments described herein generally include biasing members (such as compression springs or leaf springs), it should be appreciated that carriers without biasing members are also within the scope of the present disclosure.

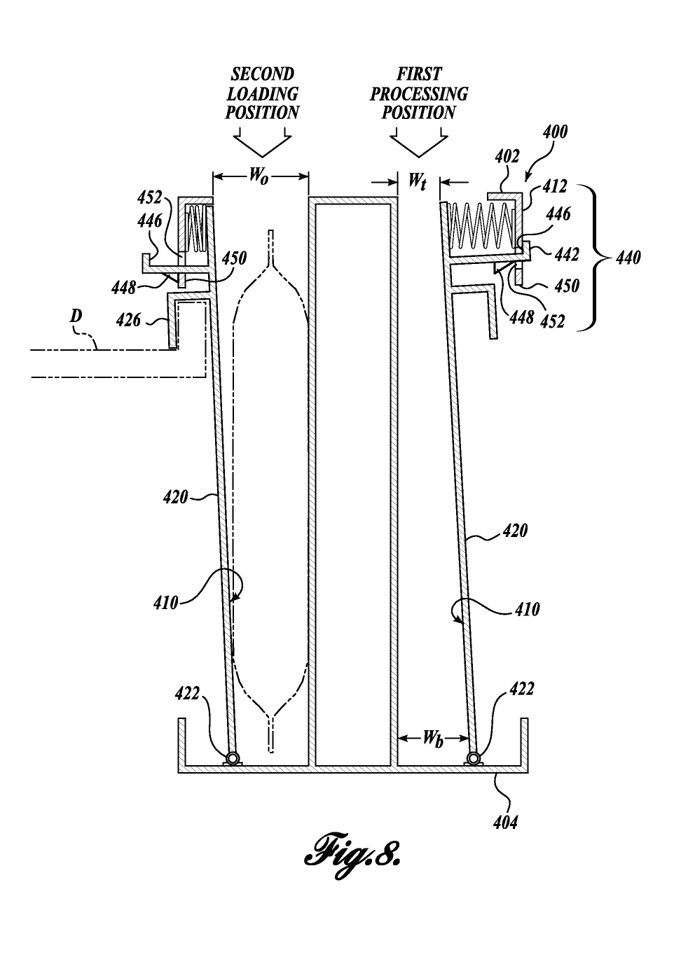

Referring now to FIGS. 11A-11D, views of an exemplary leaf spring-type carrier 700 (similar to the embodiment in FIGS. 10A and 10B) are shown. Although similar numbers are used for like parts in FIGS. 10A and 10B, it should be noted that the carrier 600 shown in FIGS. 10A and 10B is a double sleeve carrier, and the carrier 700 shown in FIGS. 11A-11C is configured as a single sleeve carrier.

The sleeve 710 for receiving a pouch (not shown) is seen in FIGS. 11A and 11B. As can be seen in FIGS. 11A and 11B, the second sleeve upright wall 720 is biased to a closed position, which can be opened by a user grasping or otherwise using handle 726.

Referring to FIG. 11D, the second sleeve wall 720 (shown as a leaf spring-type wall) is removable from the carrier 700. This type of removable wall allows for modification to the carrier 700, for example, to exchange parts (such as sleeve walls) to accommodate other types of pouches having varying size or shape, or for maintenance or cleaning. In the embodiment shown, the removable spring wall 720 includes a base 760 that is attachable to an end 762 or other portion of the carrier 700 by coupling devices 764 (see FIG. 11A). In addition, the removable spring wall 720 of the embodiment shown is designed to have a biasing action into the closed position to hold and substantially evenly distribute the contents of a pouch having a specific size and shape within the sleeve 710. It should be appreciated, however, that other biasing designs are also within the scope of the present disclosure, for example, biasing to the open position.

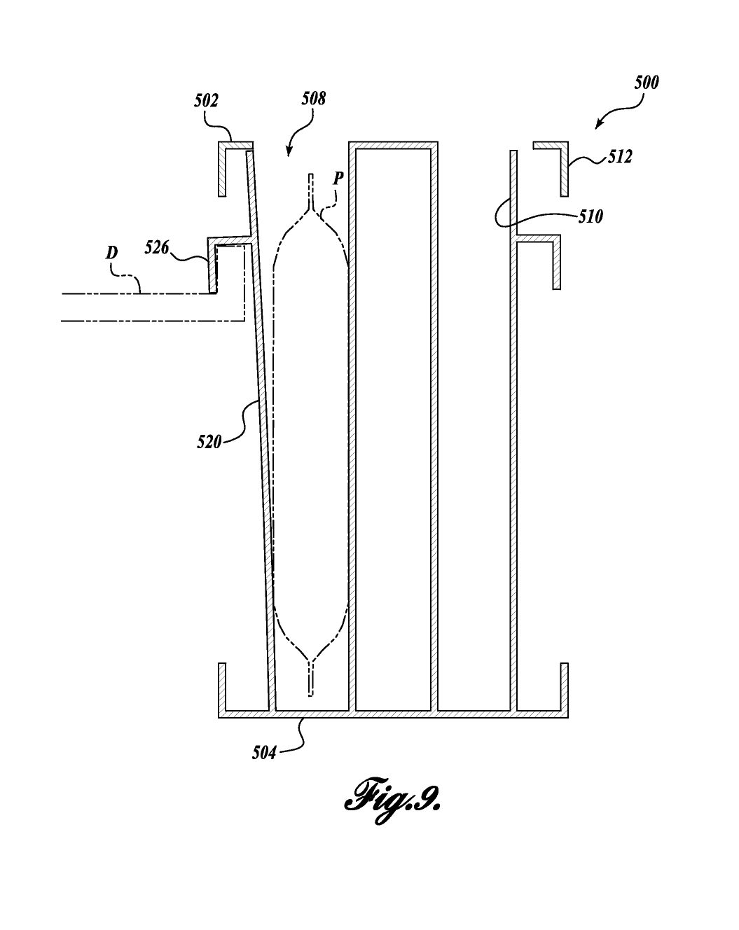

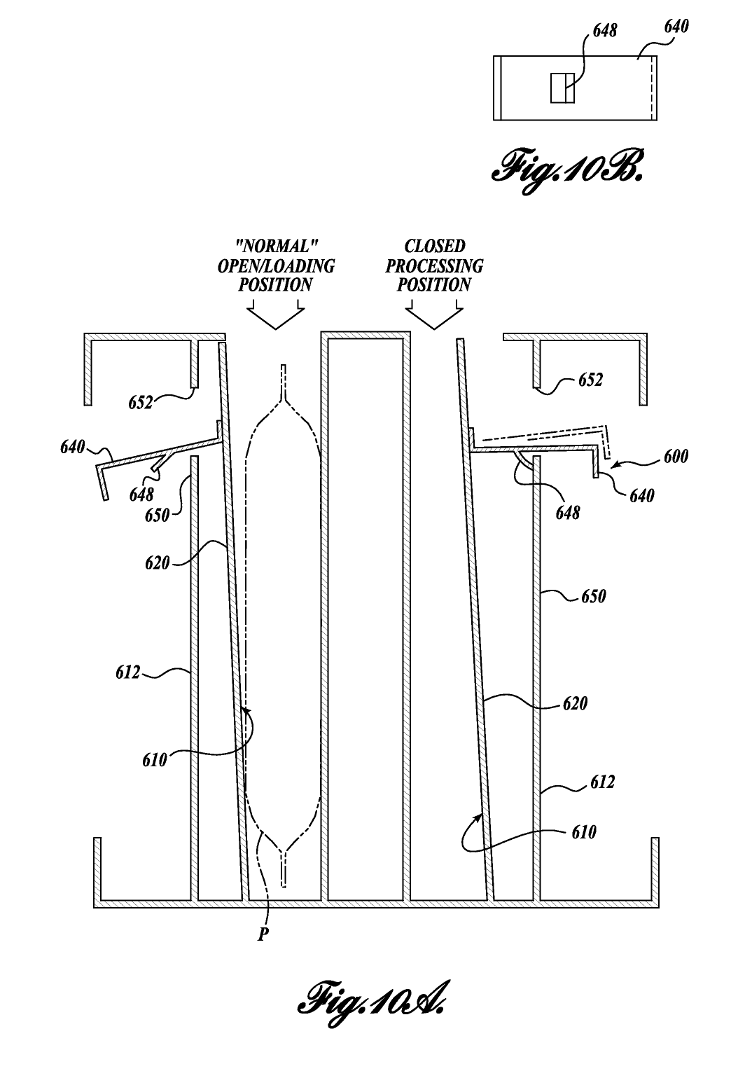

Various other embodiments of pouch retaining and release systems will now be described. Referring to FIGS. 12A-13B, instead of a wall being movable (as can be seen in the illustrated embodiments of FIGS. 6A-11D), fingers 830 and 930 extend from or through respective outer walls 820 and 920 are used to “squeeze” the pouches P. Referring to FIGS. 12A and B, the fingers 830 extend from the wall and are bent at an angle to apply compression force on the pouch P. Like the outer wall 520 shown in the illustrated embodiment of FIG. 9, the fingers 830 in the illustrated embodiment in FIGS. 12A and 12B are not hinged to the outer wall 820. Rather, they act as leaf springs, being bendable from a normal compressing position to an opened position. In that regard, the fingers may be laser cut into the outer wall, then formed with a bend configuration to provide suitable “squeeze” against the pouch P. The fingers 830 may include a handle or another pulling device (not shown) to pull them from the normal compressing position to the opened position.

In the illustrated embodiment of FIGS. 13A and 13B, the fingers 930 are attached to a hinge and torsion spring assembly 932 to rotate the fingers 930 against the pouch P. Like the illustrated embodiment of FIGS. 12A and 12B, fingers 930 may include a handle or another pulling device (not shown) to pull them from the normal compressing position to the opened position.

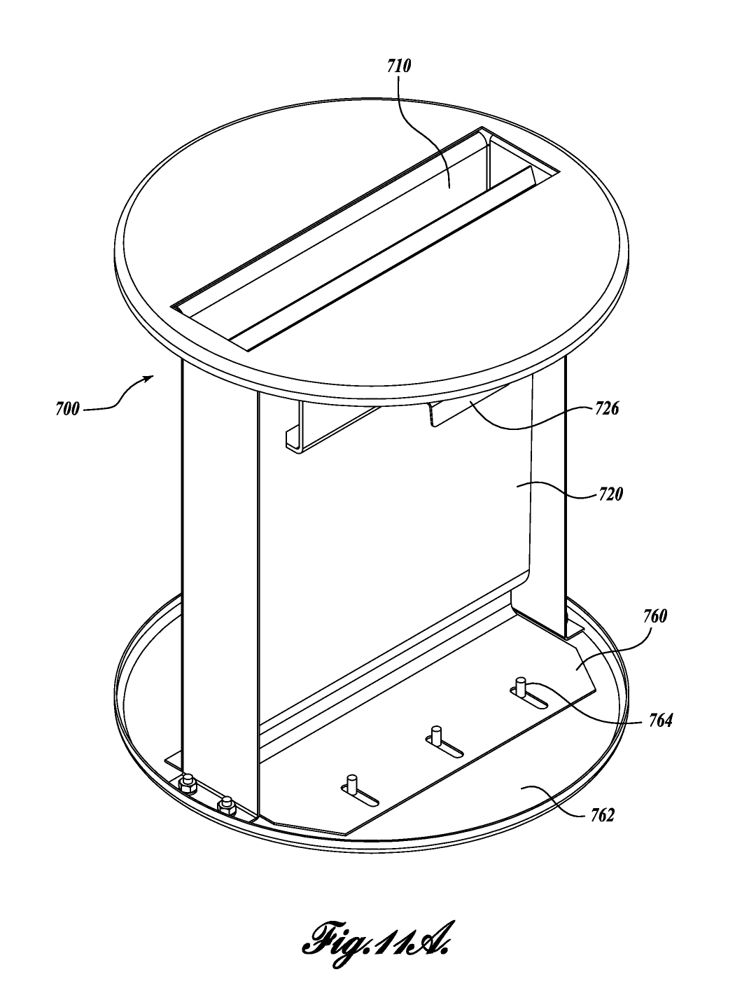

Referring to FIGS. 14A and 14B, a body portion 1012 having movable inner walls 1040 and stationary outer walls 1020 in the sleeves 1010 is shown. In that regard, the inner walls 1040 are coupled by hinges 1042 to the second end 1004 of the body portion 1012. A biasing linkage system 1044 can be used to move the inner side walls 1040 between a normal compressing position (see FIG. 14A), and an opened position (see FIG. 14B).

The linkage system 1044 generally includes a cross bar 1046 biased by a biasing member 1024 (such as a compression coil spring) into the normal compressing position (see FIG. 14A), but movable to the opened position (see FIG. 14B). The cross bar 1046 is pivotally coupled to links 1048 that are pivotably coupled to the inner walls 1040. As the cross bar 1046 is pushed down, the links 1048 pivot and push the inner walls 1040 inward. The inner walls 1040 pivot around hinges 1042 to enlarge the openings 1008 of the sleeves 1010. A pushing device (not shown) may be used to generate a force (shown as arrow F) on the cross bar 1046 to activate the biasing linkage system 1044.

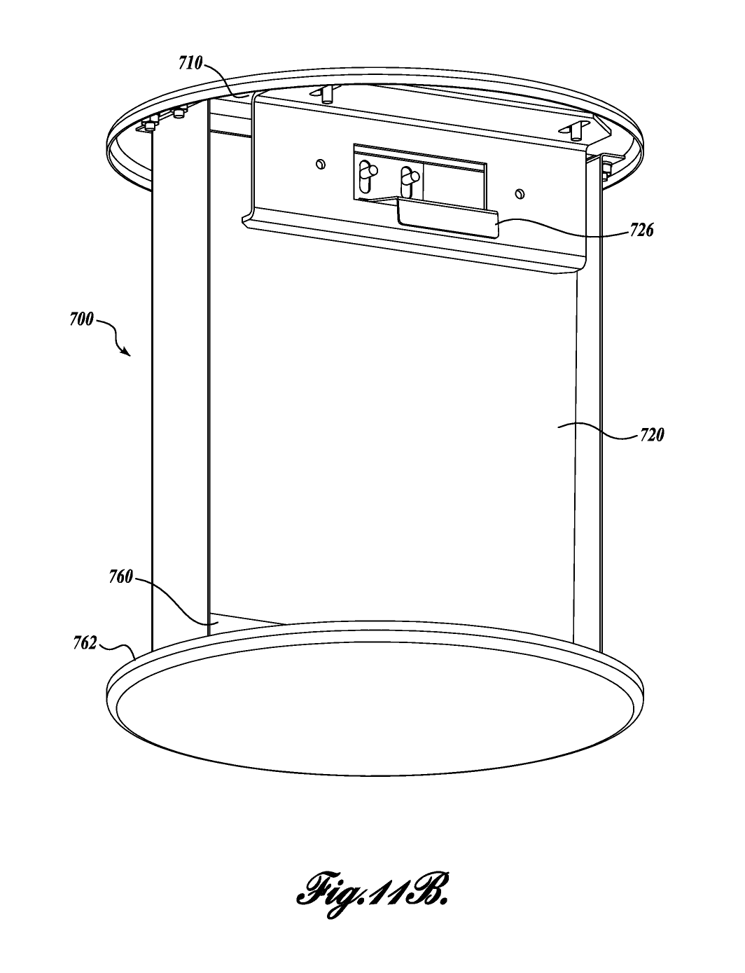

Referring now to FIGS. 15A and 15B, another embodiment of a pouch carrier 1100 having a pouch loading and retention system is shown. In the illustrated embodiment of FIGS. 15A and 15B, the substantially cylindrical pouch carrier 1100 includes first and second portions 1150 and 1152 defining first and second pockets 1154 and 1156. The first and second portions 1150 and 1152 are divided along a plane extending longitudinally through the diameter of the first and second circular ends 1102 and 1104. The first and second portions 1150 and 1152 are hingedly coupled to one another by a hinge 1160 and each includes a door 1162 and 1164 hingedly coupled so as to close each of the respective first and second pockets 1154 and 1156. A carrier latch 1166 secures the carrier 1100 in the closed position.

During pouch loading, the pouch carrier 1100 is opened to receive first and second pouches P. The pockets 1154 and 1156 are shaped to constrain pouch motion, and in that regard, may be specifically designed for certain kinds of pouches or generally designed to constrain the motion of most pouches. After the pouches P have been loaded, the doors 1162 and 1164 are closed to snuggly fit over the pouches P and constrain pouch motion. After the pouches have been loaded, at least one of the first and second portions 1150 and 1152 is rotated around hinge 1160 to latch together with latch 1166 and form a cylinder.

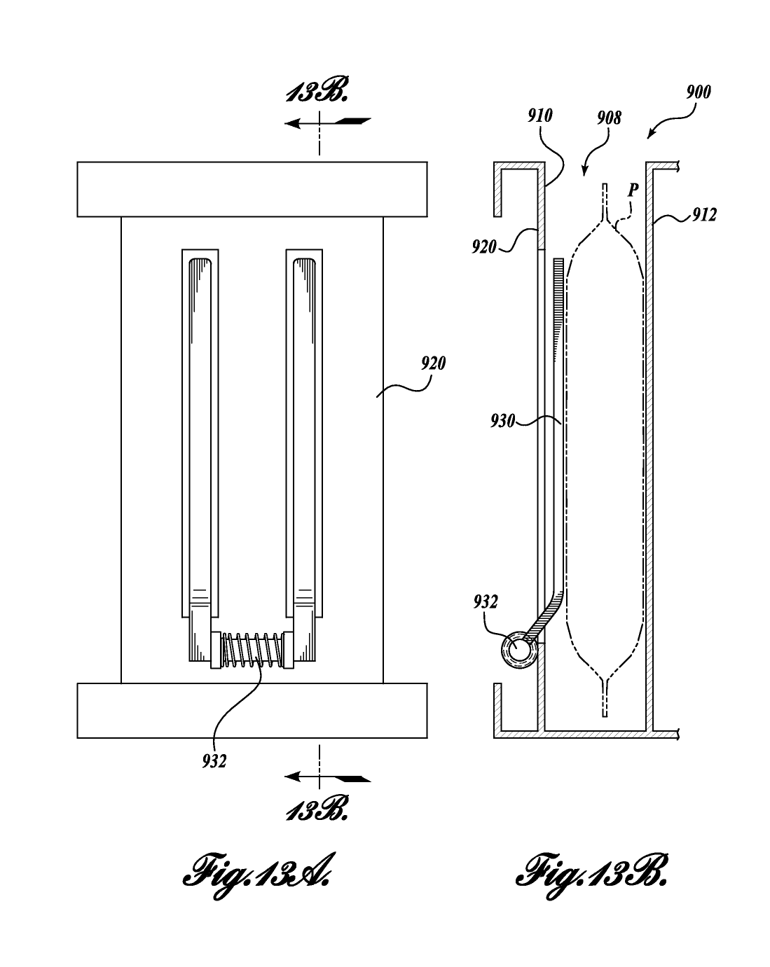

In addition to sleeve retention systems, as described above, sleeve cross-sectional shape may also assist with pouch retention to minimize movement of the pouch P within the sleeve and to more evenly distribute the contents of the pouch P throughout the entire pouch P. Referring to FIGS. 16A, 16B, and 16C, suitable cross-sectional shapes may include, but are not limited to, trapezoidal (see FIG. 16A), with tapered ends (see FIG. 16B) to accommodate the seal S along the outer peripheral edge of the pouch P (see FIG. 2), and curved (see FIG. 16C). Referring to FIG. 17, a trapezoidal sleeve shape (for example, as shown in FIG. 16A) may be combined with a hinged sleeve retention mechanism, such as that shown in FIGS. 14A and 14B.

Now referring to FIGS. 18-20 various designs for rotary systems, such as rotary sterilizers, are shown. It should be appreciated that rotary systems may be designed to handle any number of carriers at any given time. In FIG. 1, a test rotary sterilizer is shown, which is capable of handling eight carriers at any given time. Suitable larger diameter rotary sterilizers for use in production may be capable of processing thousands of carriers at any given time.

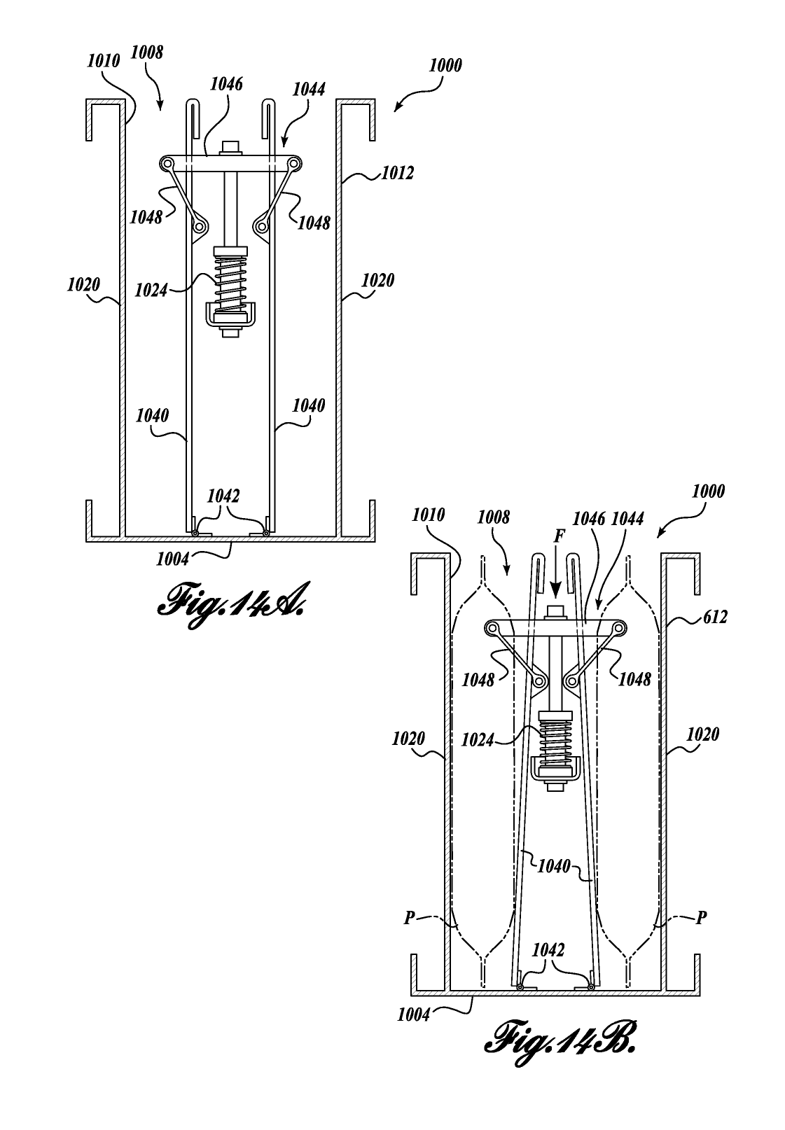

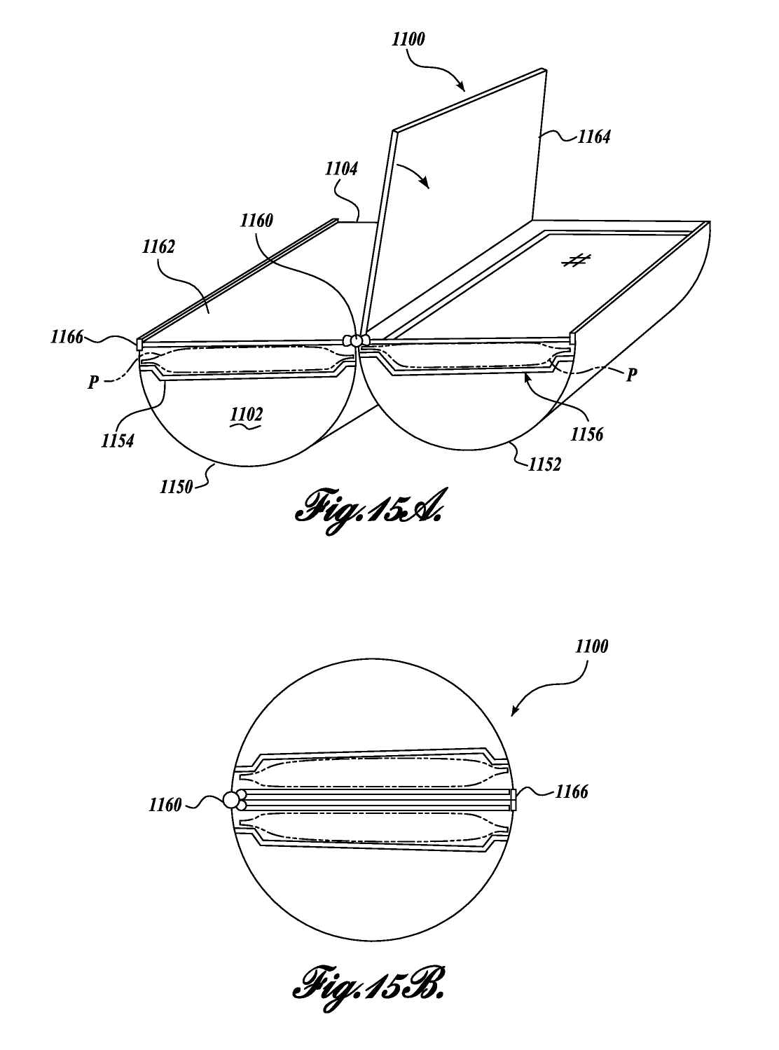

It should be appreciated that the rotary systems may have an agitating reel (see FIG. 19) or a non-agitating reel (see FIG. 20). Referring to FIGS. 18 and 19, a rotary sterilizer 1200 including an agitating reel 1204 will first be described. A rotary valve 1202 feeds the reel 1204 of the rotary sterilizer 1200 with carriers 1206. The reel 1204 is surrounded by an outer shell 1208. The carriers 1206 are rotated around the reel 1204 and are maintained in position along the reel 1204 by angles 1210 (see FIG. 19). In certain positions along the reel 1204, the carriers 1206 may be supported by the outer shell 1208. In that regard, when the carriers 1206 are on the upper portion of the reel 1204 they are supported by the reel 1204 itself, but when they are on the bottom portion of the reel 1204, they are supported by the outer shell 1208 and pushed along the outer shell 1208 by the angle 1210 located behind the carrier 1206 (see FIG. 19). When supported by the outer shell 1208, the carriers 1206 roll along the internal surface of the outer shell 1208 and therefore the carrier contents are agitated by such rolling action.

Referring now to FIG. 20, a portion of rotary system 1300 including a non-agitating reel 1304 is shown. The rotary sterilizer of the illustrated embodiment of FIG. 20 is substantially similar to the illustrated embodiment of FIGS. 18 and 19, except for a difference regarding the angles 1310. In that regard, the angles 1310 are designed to carry and support the carriers 1306 when carriers 1306 are on the bottom portion of the reel 1304. Because the carriers 1306 do not roll along the internal surface of the outer shell 1308, the carrier contents are not agitated by any rolling action.

With canned food, agitation may be desirable for certain food types, for example, milk and other low-viscosity foods. With other fragile foods, for example, lima beans, agitation may not be desired because it may cause the beans to become broken or damaged. The advantage of using a non-agitating rotary sterilizer with the many embodiments of pouch carriers described herein is that agitation is minimized, which in turn minimizes scuffing and wrinkling of the carried pouches P. Therefore, in accordance with methods of the present disclosure for processing pouches in rotary sterilizers, pouches may be placed in cylindrical carriers and received in either agitating or non-agitating rotary systems, such as rotary sterilizers.

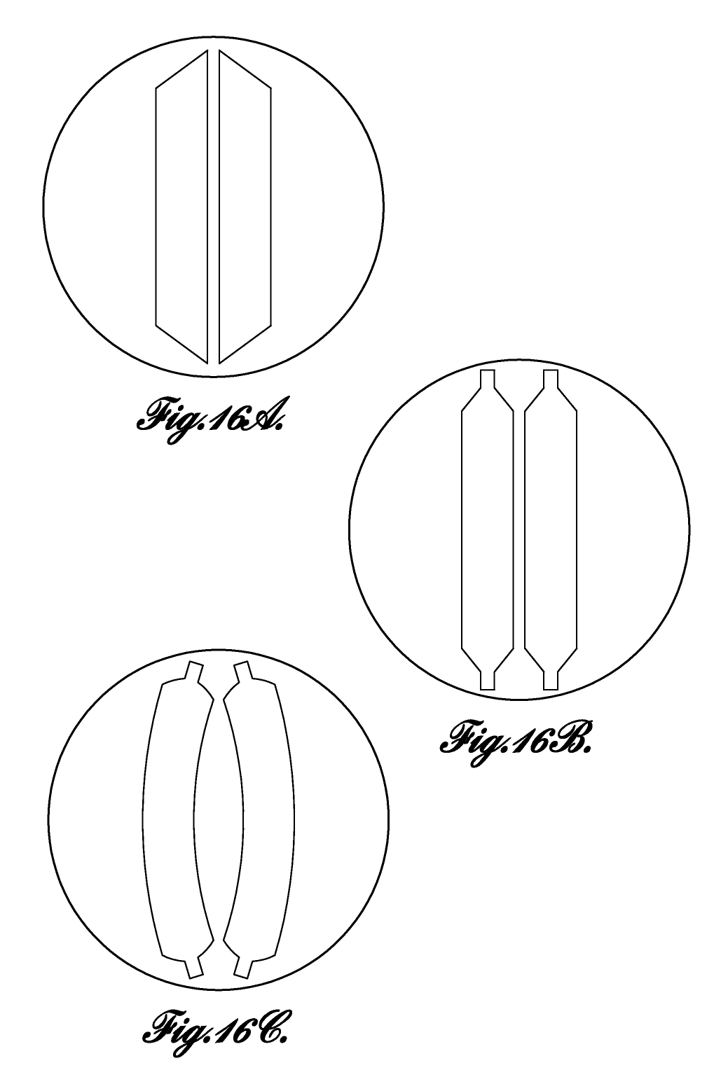

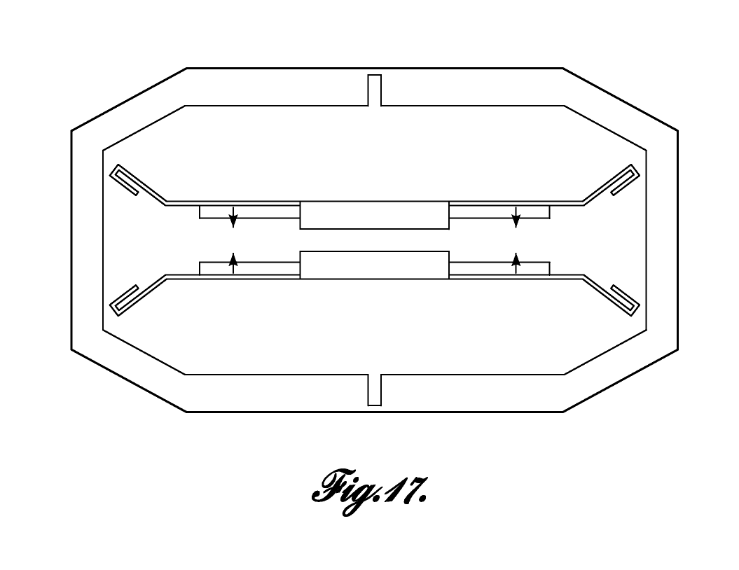

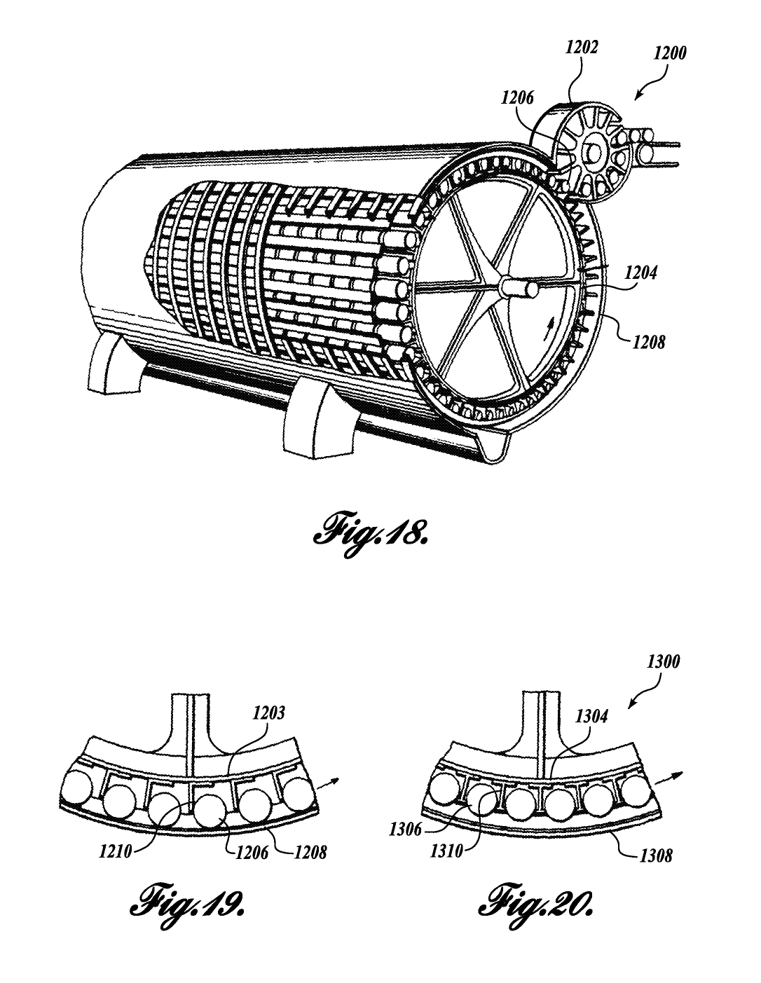

Referring to FIGS. 21-23, intermittent and continuous unloading systems 1340 and 1350, as well as a chute 1356 for receiving pouches that are dropped downwardly from a carrier, are shown. Referring to FIGS. 21 and 22, the carriers 600 are supported by two laterally spaced apart conveyors 1352 positioned far enough apart that the pocket openings of the carriers 600 are positioned over the open width 1354 between the conveyors 1352. In the illustrated embodiments of FIGS. 21-23, the carriers do not rotate on the conveyors, but rather are supported by the conveyors and/or by guide rails positioned beneath either of the first or second ends of the carrier (see, e.g., FIG. 23 showing conveyors 1352 positioned beneath carrier 600). This support system maintains the carriers in a non-rotating position, preferably with the openings to the sleeves oriented downwardly.

In addition to conveyors 1352 for support, the systems 1340 and 1350 may also include an optional alignment feature. Using an alignment feature, the carrier must be rotated or otherwise indexed to a correct position for loading or unloading pouches. For example, using the geometry of the carrier, if the central box containing the sleeves is rectangular in shape, a carrier can thus be guided along conveyors such that the long side of the box is parallel to travel. The orientation of the pocket openings is thus known. In the illustrated embodiment of FIG. 23, guide rails 1358 run the length of the conveyors 1352, are parallel to the conveyors 1352, and are configured for maintaining the carriers 600 on the conveyors 1352. In that regard, the carriers 600 may be top heavy, and the guide rails 1358 will prevent them from toppling over.

At the appropriate time, the carriers 600 are acted upon to allow the pouches P contained in the sleeves to drop downwardly from the sleeves (largely by gravity). Referring to FIGS. 21 and 23, pulling devices D (for example, as shown and described with reference to FIGS. 8 and 9) may be activated to move a carrier wall from a pouch retaining position to a pouch release position. Referring to FIG. 22, a pulling bar B may be positioned along the path of the conveyors 1352 to move a carrier wall from a pouch retaining position to a pouch release position. Such a pulling bar may be active, as described with reference to FIGS. 21 and 23, or it may be passive, for example, using a stationary bar with sloped surfaces that “cam” the carrier wall to the pouch release position as the carrier 600 moves along the conveyors 1352.

The pouches P will travel between the two supporting conveyors 1352 downwards onto appropriate chutes 1356 or take-away conveyors (see, e.g., FIG. 23). The action of unloading and dropping pouches could occur while the carrier is either stopped (for example, at an “unload position” showing in FIG. 21), or is moving along the support conveyors (for example, see “unload zone” in FIG. 22). It should be appreciated that a continuous unloading process may allow for faster system operating conditions by reducing carrier stoppages.

Referring to FIGS. 21 and 22, mechanisms can be activated at the “unload position” 1360 or “unload zone” 1370 to actuate a latch mechanisms and open the sleeve of the container 600. To allow integration with a full processing system, such as loading, unloading, and controls, other features can be included in the carrier design, as described in greater detail below. As one non-limiting example, the carriers may include a “pouch present” feature. Such a feature may includes holes in the walls of the carrier to allow photo eyes (for example) to shine through the carrier to verify if the pocket does or does not contain a pouch.

As another non-limiting example, the carriers may include “sleeves up” feature for determining if the carrier is correctly oriented for loading or unloading: sleeves upward, or downward, as required. For example, the pocket openings can be sensed using eddy current proximity sensors, magnetic sensors, or other appropriate sensors, and appropriate action can be taken to remove incorrectly oriented carriers.

It should be appreciated that the carrier unloading processes of FIGS. 21-23 may be adapted for use with any of the above-described embodiments carriers. However, the carriers illustrated in FIGS. 21-23 generally include release mechanisms for unloading pouches (such as position limiter assemblies shown in FIGS. 10A and 10B).

In accordance with these processes, carriers (as described above) can be unloaded after processing by rotating or otherwise orienting the carriers with the openings of the sleeves directed downwards, and aligning the carrier so that the long side of the sleeve is, for example, parallel to the path of travel along a conveyor. Likewise, carriers can be loaded by orienting the carriers with the sleeves upwards, aligning the carrier so that the long side of the sleeve is, for example, parallel to the path of travel along a conveyor.

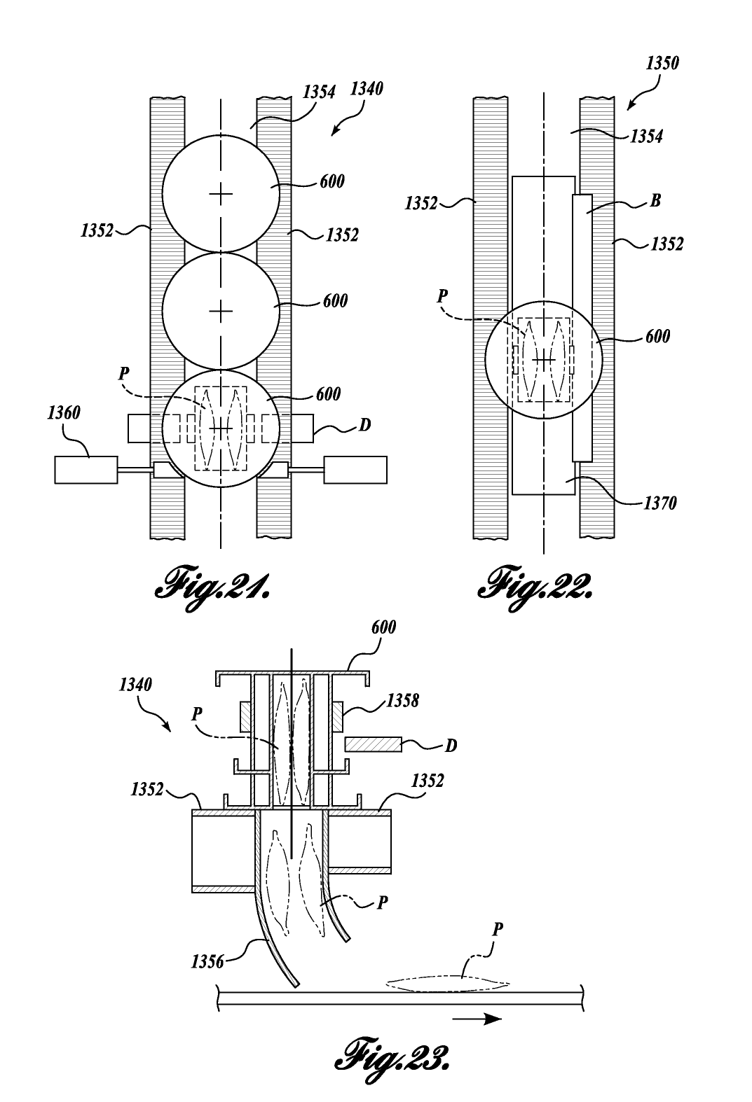

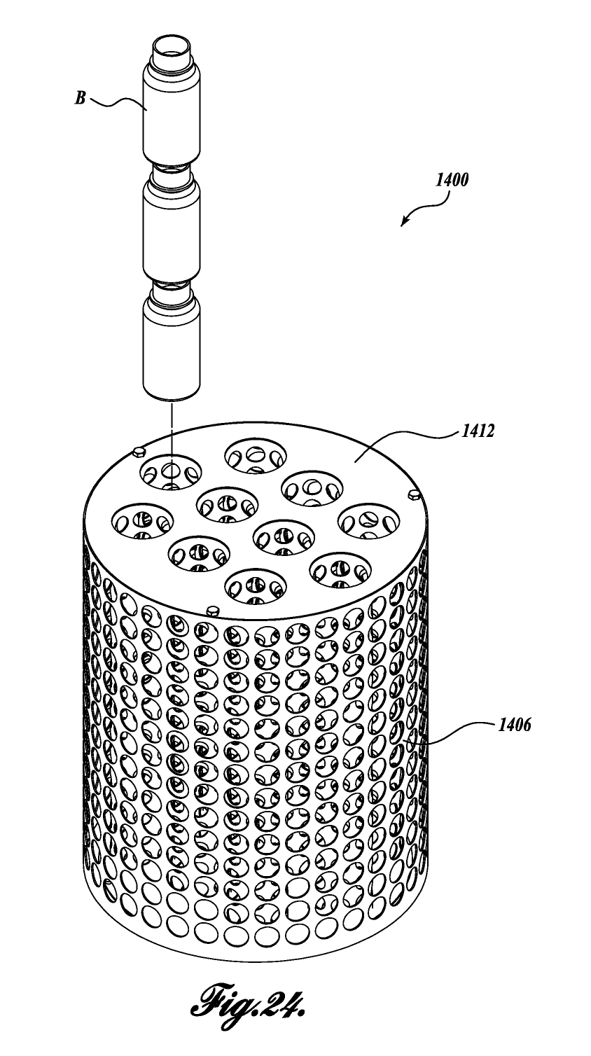

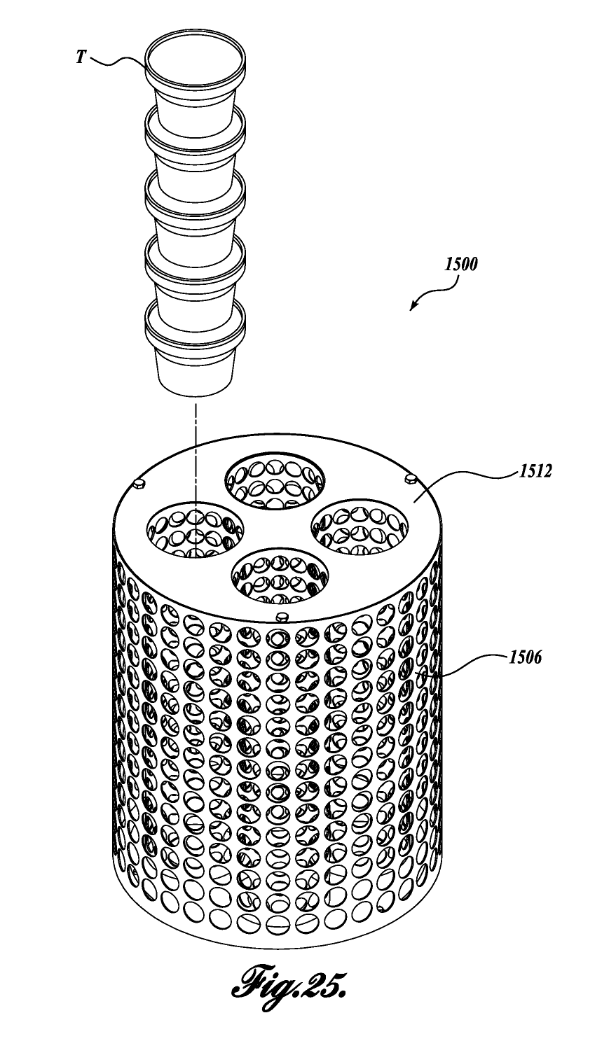

Now referring to FIGS. 24-27, other carrier embodiments are shown for carrying other types of containers besides pouches. In that regard, FIG. 24 is a bottle carrier 1400, FIG. 25 is a tub carrier 1500, and FIG. 26 is a fiberboard and plastic laminate container carrier 1600, all in accordance with embodiments of the present disclosure. In accordance with embodiments of the present disclosure, the carriers 1400, 1500, and 1600 of respective FIGS. 24-27 may include suitable retention systems (see e.g., the retention system shown in FIG. 28).

Referring to FIG. 24, carrier 1400 includes a body portion 1412 having first and second ends, and an outer shell portion 1406. The body portion 1412 includes a plurality of sleeves for receiving containers, such as bottles. The shell and body portion 1406 and 1412 may be perforated to allow for effective heat transfer. In the illustrated embodiment, the shell portion 1406 is substantially cylindrical and the first and second ends are substantially circular to allow the carrier 1400 to roll. Referring now to FIGS. 25 and 26, the respective carriers 1500 and 1600 are substantially similar to the carrier 1400 of FIG. 24, but include different sized or shaped sleeves for receiving, for example, tubs and laminate containers. Any of the carriers 1400, 1500, and 1600 of respective FIGS. 24-26 may also be configured for receiving pouches.

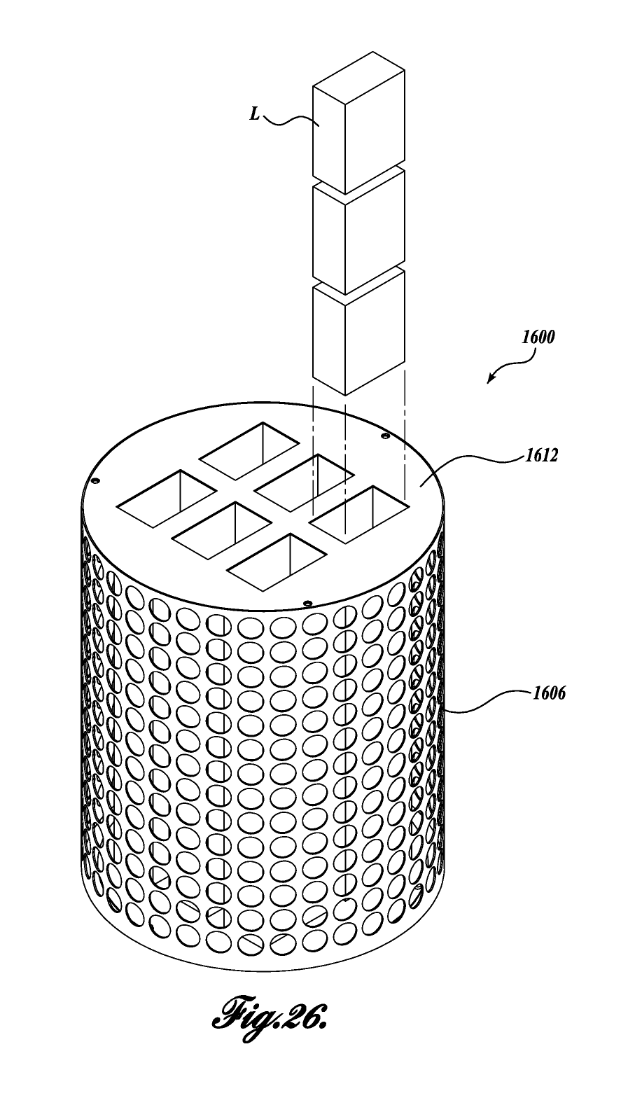

As seen in FIG. 27, the body portions 1412, 1512, and 1612 of the respective bottle, tub, and laminate container carriers 1400, 1500, and 1600 may each be interchangeable within a single outer shell portion 1406. Such interchangeability of parts allows for various types of containers to be processed using the carrier system, as well as replacement of worn parts without requiring replacement of the entire carrier.

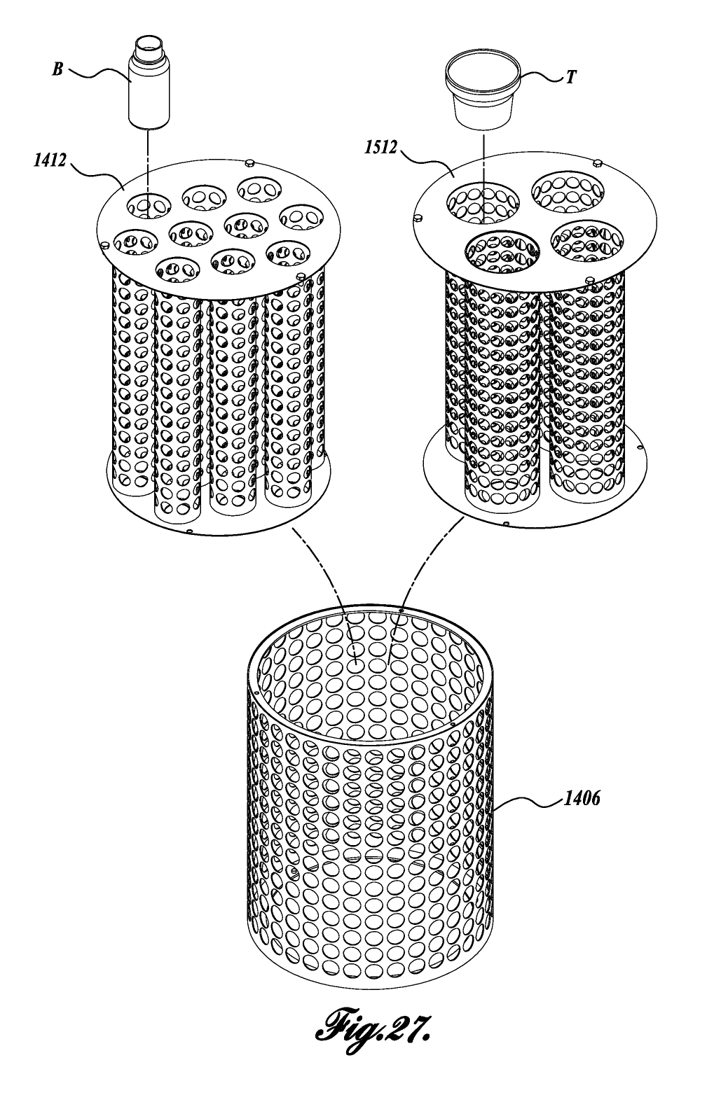

Referring now to FIGS. 28 and 29, alternate embodiments for carrier assemblies are provided. The carriers 1700 and 1800 are substantially cylindrical in shape, like the previously described embodiments; however, they have differences regarding the positioning and orientation of the sleeves 1710 and 1810 for carrying pouches P. For example, as shown in FIG. 28, the sleeves 1710 are positioned to extend radially from the center longitudinal axis of the container 1700. An optional circular-shaped capping portion 1714 having a plurality of holes 1712 can be fixed to the top surface 1716 of the container 1700. After pouches P have been received in the sleeves 1710 through aligned holes 1712 in the capping portion 1714, the capping portion 1714 may be turned relative to the container 1700 and sleeves 1710 to misalign the holes 1712 in the capping portion 1714 with the sleeves 1710. Such misalignment helps maintain the pouches P in the sleeves 1710 during processing.

Referring now to FIG. 29, the sleeves 1810 are also positioned to extend radially from the center longitudinal axis of the container 1800. However, different from the embodiment shown in FIG. 28, the sleeves 1810 are accessed from the outer cylindrical surface 1816 of the container 1800. An optional cylindrical capping portion 1814 having a plurality of holes 1812 can be fixed to the outer cylindrical surface 1816 of the container 1800. After pouches P have been received in the sleeves 1810 through aligned holes 1812 in the capping portion 1814, the capping portion 1714 may be turned relative to the container 1800 and sleeves 1810 to misalign the holes 1812 in the capping portion 1814 with the sleeves 1810. Such misalignment helps maintain the pouches P in the sleeves 1810 during processing.

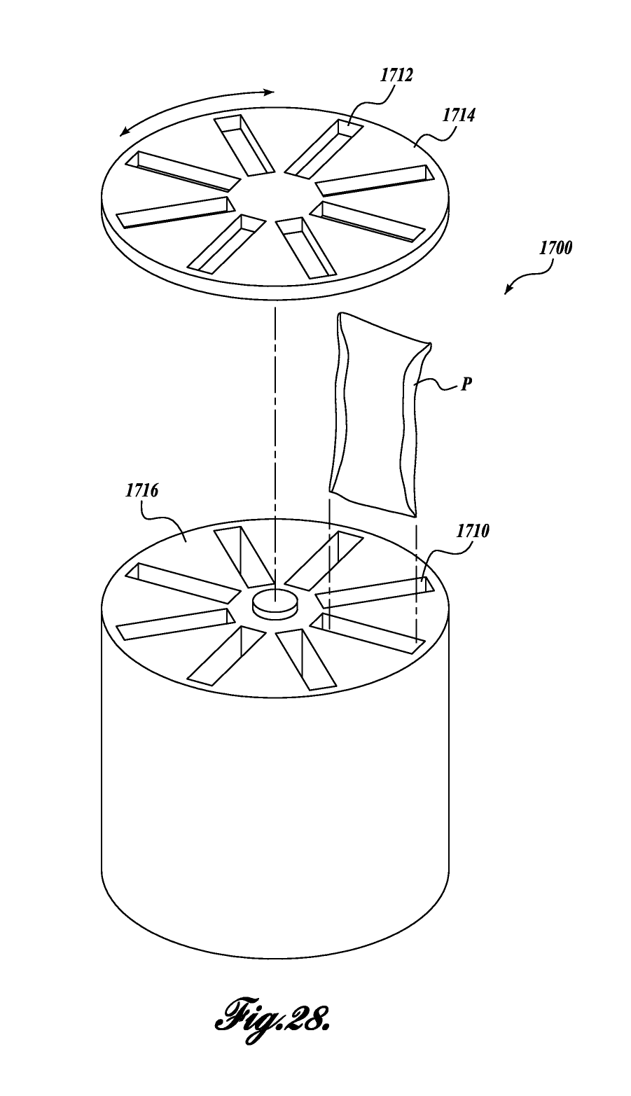

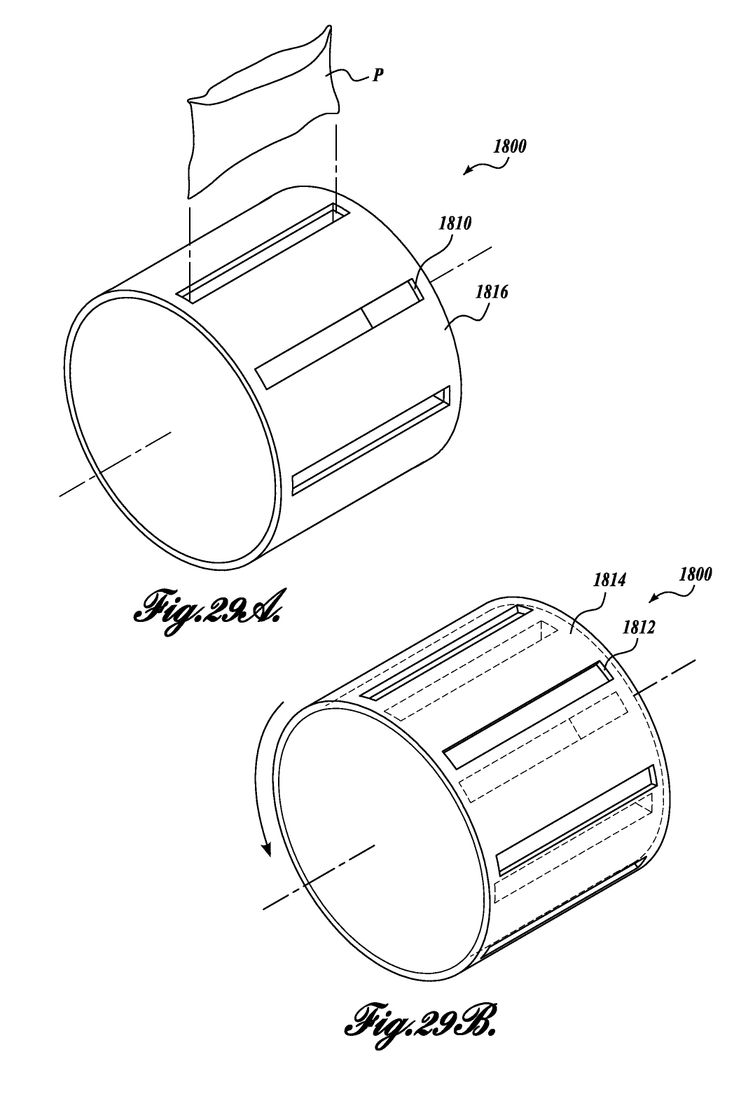

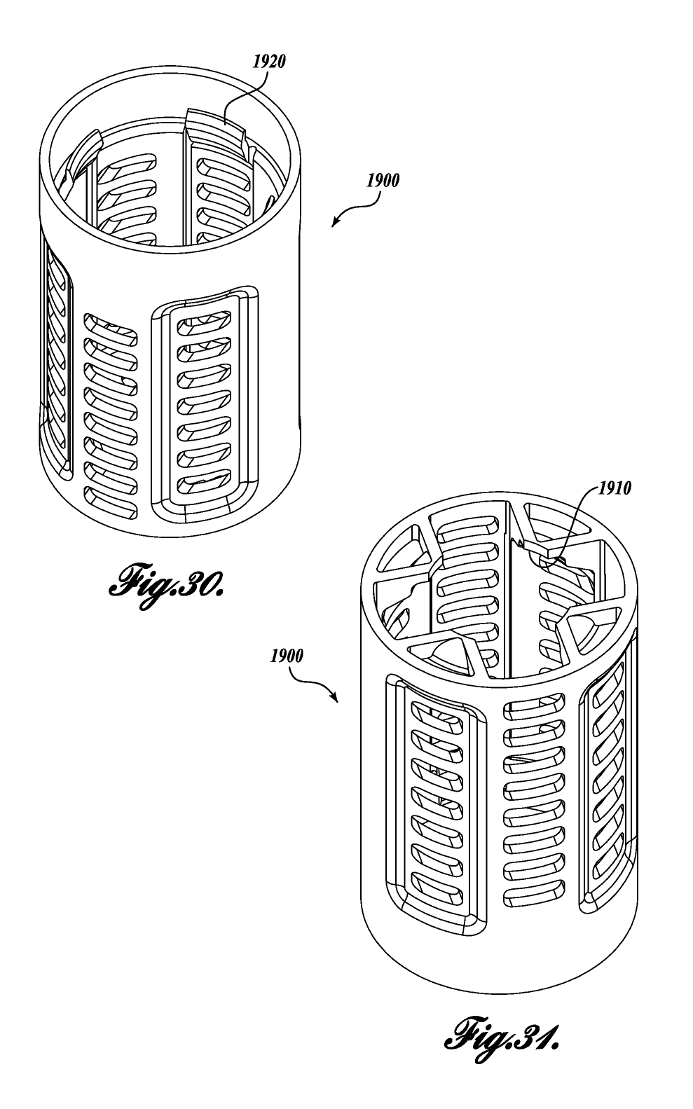

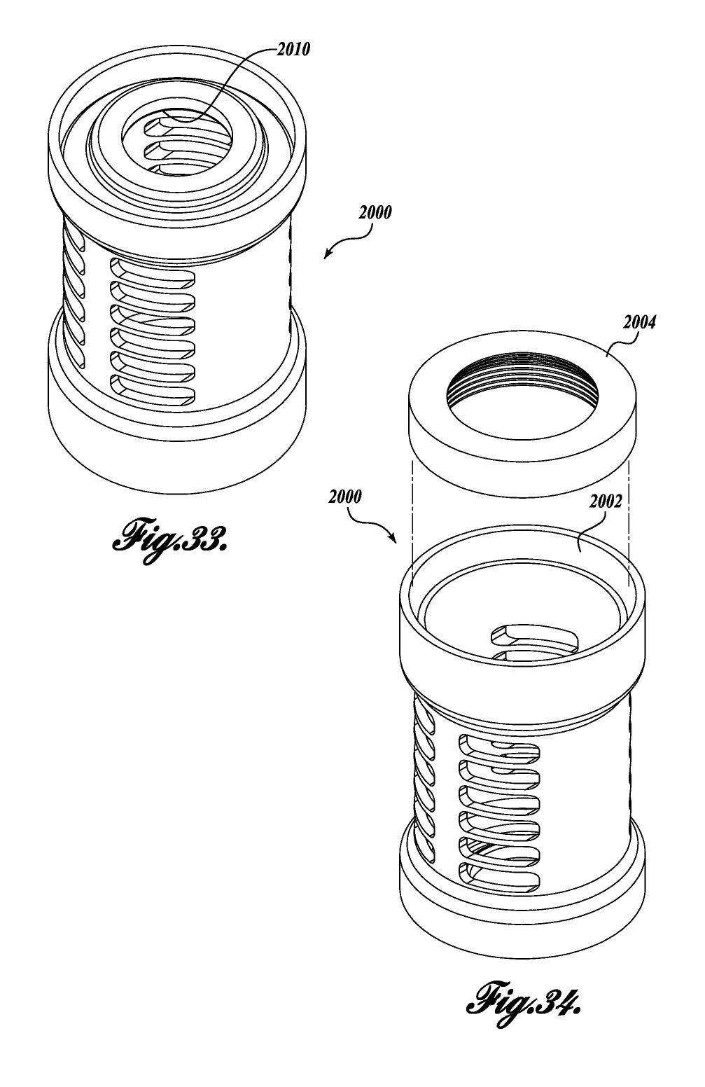

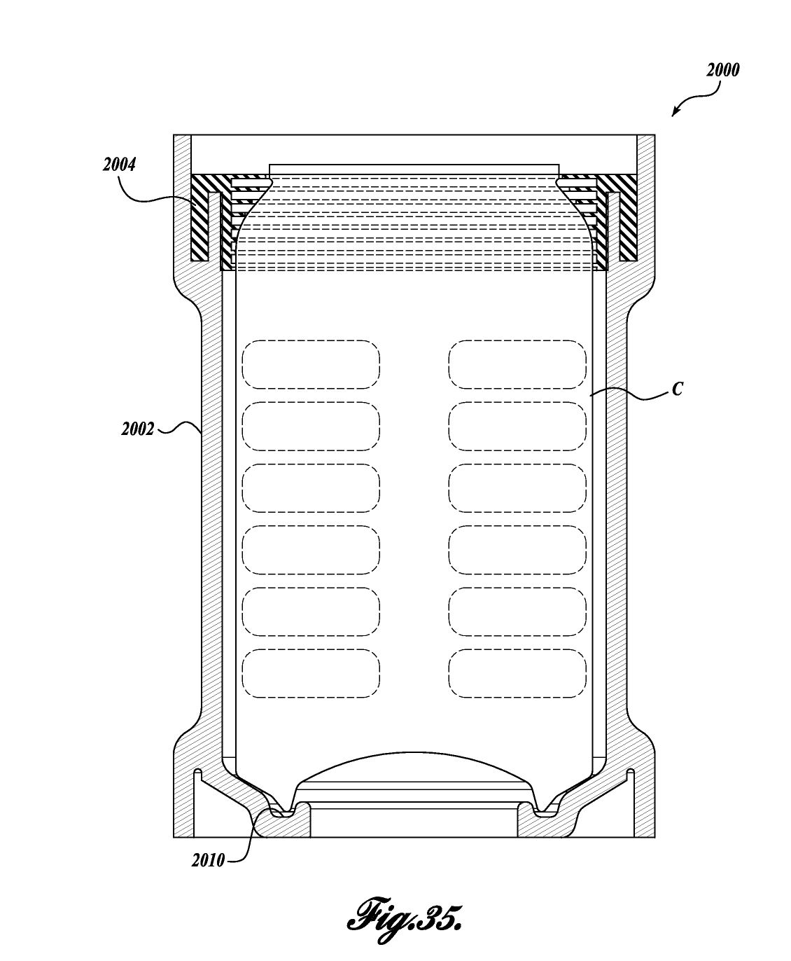

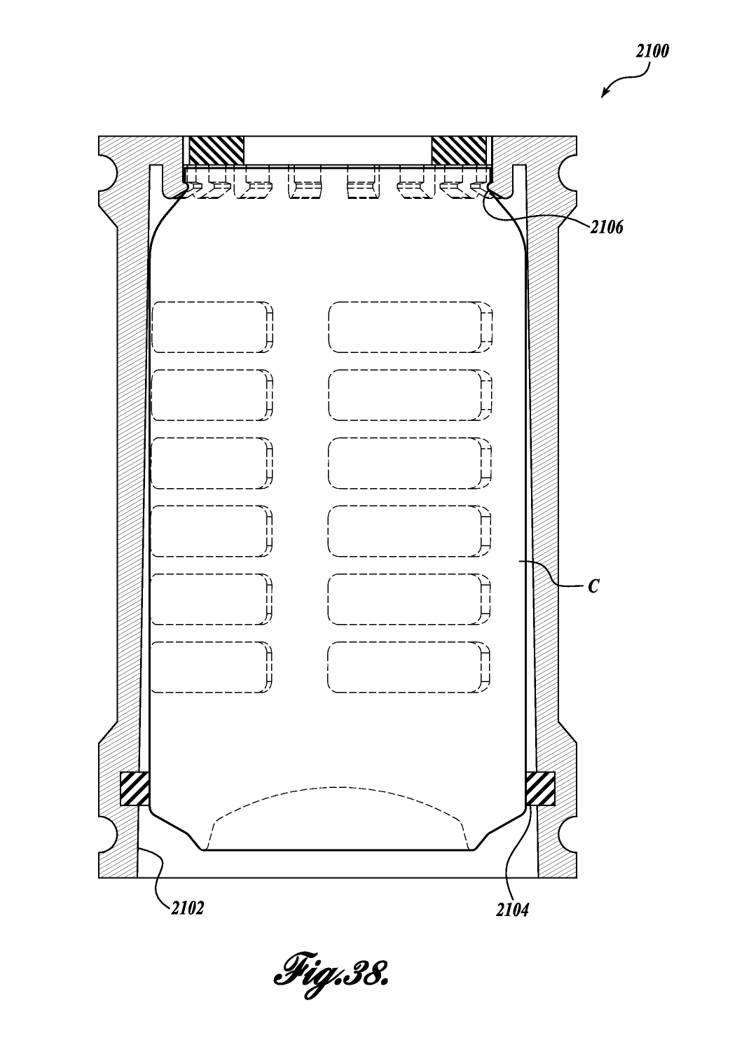

Now referring to FIGS. 30-38, various carrier embodiments are shown for carrying fragile or other irregularly-shaped containers, such as thin-walled cans, shaped cans, and shaped plastic bottles. Thin-walled cans are particularly fragile containers that can be damaged during continuous processes. The advantages of such carriers are as follows: (1) to prevent damage to the graphics on the cans; (2) to hold the can so that it will not be released from the carrier, and also to restrict movement to prevent rubbing; (3) to maximize the heat penetration through the carrier so that it reaches the contents inside the can; and (4) to handle irregular-shaped containers as though they are cylinders.

Referring to FIGS. 30-32, a carrier 1900 in accordance with one embodiment of the present disclosure is shown. The carrier 1900 is designed from molded plastic, such as polypropylene. The carrier 1900 may be made from any suitable process, for example, injection molding process. Holes along the exterior surface of the carrier 1900 may be used to meeting molding parameters or reduce material usage, as well as provide access for processing fluids.

The design generally includes a shaped area 1910 in the bottom of the carrier 1900 for mating with the contoured bottom end of a typical two-piece can, and plastic tabs 1920 at the upper end for mating with the seam chimes or other geometry on the top end of a typical can. The points of contact are therefore on the top and bottom ends of the can, to prevent rubbing, denting, or other damage to the seam chimes and the outer cylindrical wall of the can. This carrier 1900 can also be used with a two-piece can, can loaded top-end first; with three-piece cans having either end being captured by plastic tabs; or with plastic bottles having tabs designed to capture an appropriate feature on the bottle.

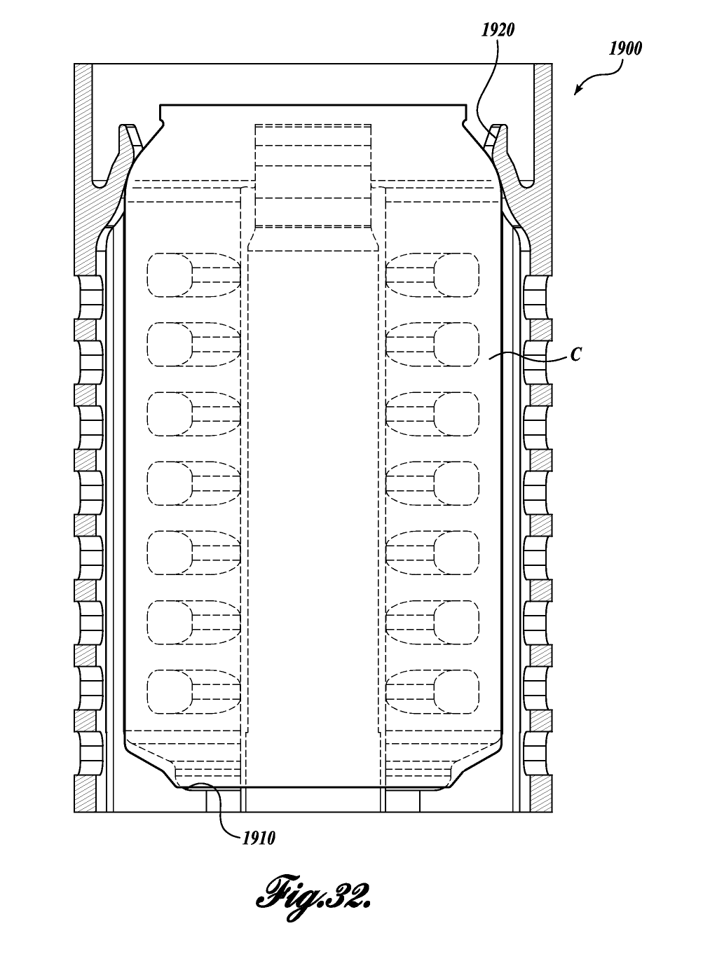

Referring to FIGS. 33-35, a carrier 2000 in accordance with another embodiment of the present disclosure is shown. The carrier 2000 includes a main receiving portion 2002 and an insert portion 2004 for mating with the main receiving portion 2002. The insert portion 2004 may be formed from a compliant material, such as rubber or silicon. Like the carrier 1900 shown in FIGS. 30-32, the design includes a shaped area 2010 in the bottom of the carrier 2000 for mating with the contoured bottom end of a typical two-piece can. After the can has been received in the carrier 2000, the insert portion 2004 engages with the main receiving portion 2002 to hold the can in the carrier 2000.

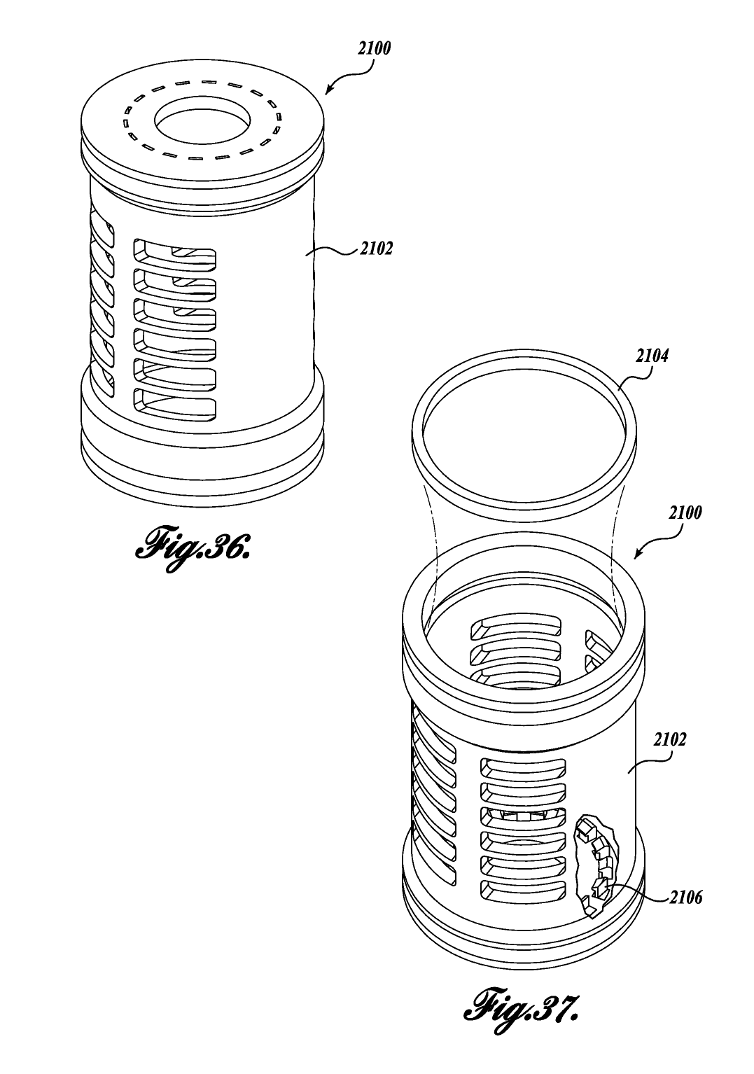

Referring to FIGS. 36-38, a carrier 2100 in accordance with another embodiment of the present disclosure is shown. The carrier 2100 includes a main receiving portion 2102 and an insert portion 2104 for mating with the main receiving portion 2102. The insert portion 2104 may be formed from rubber or silicon or another suitable material having compressibility and flexibility properties. The bottom or receiving portion 2102 incorporates tabs 2106 that capture a feature on the can, such as the seam chimes, to retain the can in the carrier 2100. An appropriate feature on a container is a groove that the tabs 2106 can fit into to restrict the container from falling out and to support the fragile surfaces. For cans, this feature may be the necked-in diameter adjacent the seam chimes For a plastic bottle, this feature may be the tapered upper shoulder that approaches the bottle neck. The tabs 2106 are preferably flexible enough to deflect when the container is inserted into the carrier 2100, but then to flex back to into position when the container is removed from the carrier 2100.

While illustrative embodiments have been illustrated and described, it will be appreciated that various changes can be made therein without departing from the spirit and scope of the disclosure.

1. A carrier for a container for processing the container in a rotary processing system, the carrier comprising:
(a) a carrier body defining at least one compartment for receiving at least one container, wherein the carrier is capable of rolling; and
(b) a retention system for maintaining the at least one container in the at least one compartment and restricting movement of the at least one container within the at least one compartment.

2. The carrier of claim 1, wherein the carrier is substantially cylindrical.

3. The carrier of claim 1, wherein the carrier has substantially circular ends.

4. The carrier of claim 1, wherein the retention system includes a retention device and biasing member for biasing the retention device between a retaining position and a loading position.

5. The carrier of claim 1, wherein the retention system does not separate from the carrier body.

6. The carrier of claim 4, wherein the biasing member is normally biased in the retaining position.

7. The carrier of claim 4, wherein the biasing member is normally biased in the loading position.

8. The carrier of claim 4, wherein the biasing member is a leaf spring.

9. The carrier of claim 4, wherein the biasing member is a spring.

10. The carrier of claim 1, wherein the retention system further includes a hinge about which the retention device rotates between retaining and loading positions.

11. The carrier of claim 1, wherein the retention system includes a position limiting assembly.

12. The carrier of claim 11, wherein the position limiting assembly may be fixed in at least a first position.

13. The carrier of claim 11, wherein the position limiting assembly may be fixed in at least first and second positions.

14. The carrier of claim 11, wherein the position limiting assembly may be fixed in at least first, second, and third positions.

15. The carrier of claim 1, wherein the at least one container is a flexible container having movable contents.

16. The carrier of claim 1, wherein the retention system provides resistance against the at least one container to distribute the movable contents substantially evenly throughout the at least container.

17. The carrier of claim 1, wherein the at least one container is a fragile container.

18. The carrier of claim 1, wherein the at least one container is an irregularly shaped container.

19. The carrier of claim 1, wherein the compartments are sleeves.

20. The carrier of claim 1, wherein the retention system includes first and second compartments hingedly coupled to one another between open and closed positions and defining a cylinder in the closed position.

21. The carrier of claim 1, wherein the carrier body has a body portion and first and second ends, and wherein the retention system only interfaces with the first and second ends of the at least one container.

22. A carrier for a container for processing the container in a rotary processing system, the carrier comprising:
(a) a carrier body defining at least one compartment for receiving at least one container; and
(b) a removable outer shell for the carrier body.

23. The carrier of claim 22, wherein the compartments are configured for receiving one or more containers selected from the group consisting of bottles, tubs, pouches, and laminate containers.

24. A method of loading a carrier with a container, the method comprising:
(a) obtaining a carrier defining at least one compartment for receiving at least one container and a retention system for maintaining the at least one container in the at least one compartment;
(b) loading the at least one container in the at least one compartment; and
(c) activating the retention system to restrict the movement of the at least one container within the at least one compartment.

25. The method of claim 24, further comprising deactivating a retention system prior to loading the at least one container in the at least one compartment.

26. A method of loading a carrier with a container, the method comprising:
(a) obtaining a carrier defining first and second compartments hingedly coupled to one another between open and closed positions and defining a cylinder when in the closed position;
(b) placing the first and second compartments in the open positions and receiving at least one container in either of the first and second compartments;
(c) restricting the movement of the at least one container within either of the first and second compartments; and
(d) hingedly coupling the first and second compartments in the closed position to define a carrier.

27. A method of processing a container in a rotary processing system, the method comprising:
(a) placing at least one container in a carrier; and
(b) receiving the at least one carrier in the rotary system.

28. The method of claim 27, wherein the rotary system includes an agitating reel.

29. The method of claim 27, wherein the rotary system includes a non-agitating reel.

30. The method of claim 27, wherein the rotary system is a rotary sterilizer.

31. A system for unloading a container from a carrier having at least one compartment for receiving at least one container and a retention system for maintaining the at least one container in the at least one compartment, the system comprising:
(a) a conveyor assembly for conveying the carrier in a controlled path; and
(b) an unloading assembly for opening the at least one compartment.

32. The system of claim 31, wherein the conveyor assembly supports the carrier by supporting at least one of the first and second ends of the carrier.

33. The system of claim 31, wherein the conveyor assembly does not rotate the carrier.

34. The system of claim 31, further comprising a container receiving assembly for receiving the at least one container from the opened at least one compartment.

35. The system of claim 31, wherein the conveyor system includes first and second laterally spaced-apart conveyors.

36. The system of claim 31, wherein the compartment has an openable end, and wherein the carrier is received with the openable end directed substantially downward.

37. The system of claim 31, wherein the unloading assembly deactivates the retention system.

38. The system of claim 31, wherein gravity assists to release the at least one container from the at least one compartment.

39. The system of claim 31, wherein the unloading assembly is an intermittent unloading assembly.

40. The system of claim 31, wherein the unloading assembly is a continuous unloading assembly.

41. The system of claim 31, further comprising a receiving assembly for receiving the at least one container.

42. A method for unloading a container from a carrier, the method comprising:
(a) receiving on a conveyor assembly, a carrier having at least one compartment for receiving at least one container and a retention system for maintaining the at least one container in the at least one compartment, wherein the conveyor assembly is configured for conveying the carrier in a controlled path; and
(b) opening the at least one compartment.

43. The method of claim 42, wherein the conveyor assembly supports the carrier by supporting at least one of the first and second ends of the carrier.

44. The method of claim 42, wherein the conveyor assembly does not rotate the carrier.

45. The method of claim 42, further comprising receiving the at least one container from the opened at least one compartment.
